SEQUENCE LISTING (13 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg

SEQ ID NO: 5 - DNA, 24 bp, synthetic construct
  source 1..24: mol_type = other DNA; note = PX primer
aggaggaggaggaggaggaggagg

SEQ ID NO: 6 - DNA, 30 bp, synthetic construct
  source 1..30: mol_type = other DNA; note = PY primer
gaagaagaagaagaagaagaagaagaagaa

SEQ ID NO: 7 - DNA, 24 bp, synthetic construct
  source 1..24: mol_type = other DNA; note = cPX primer
cctcctcctcctcctcctcctcct

SEQ ID NO: 8 - DNA, 30 bp, synthetic construct
  source 1..30: mol_type = other DNA; note = cPY primer
ttcttcttcttcttcttcttcttcttcttc

SEQ ID NO: 9 - protein, 13 aa, synthetic construct
  source 1..13: note = SpyTag: amino acid sequence
AHIVMVDAYKPTK

SEQ ID NO: 10 - protein, 134 aa, synthetic construct
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK

SEQ ID NO: 11 - protein, 5 aa, synthetic construct
  source 1..5: note = SpyCatcher amino acid sequence
LPXTG

SEQ ID NO: 12 - protein, 4 aa, synthetic construct
  source 1..4: note = Sortase-coupling
GGGG

SEQ ID NO: 13 - protein, 5 aa, synthetic construct
  source 1..5: note = Sortase-coupling
GGGGG

(19) United States
(12) Patent Application Publication
(10) Pub. No.: US 2025/0101505 A1
(43) Pub. Date: Mar. 27, 2025

(54) CAPTURING AND AMPLIFYING POLYNUCLEOTIDES USING DENDRITIC MOLECULES

(71) Applicant: Illumina, Inc., San Diego, CA (US)

(72) Inventors: Nam Trong Nguyen, Cambridge (GB); Xavier von Hatten, Cambridge (GB); Xiangyuan Yang, Singapore (SG); Eric Brustad, San Diego, CA (US); Will Tovey, Bury St Edmunds (GB); Matthew Worsdale, Cambridge (GB); Gianluca Artioli, Cambridge (GB); Teodora Gal, London (GB)

(73) Assignee: Illumina, Inc., San Diego, CA (US)

(21) Appl. No.: 18/894,517

(22) Filed: Sep. 24, 2024

Related U.S. Application Data
(60) Provisional application No. 63/585,544, filed on Sep. 26, 2023.

(51) Int. Cl. C12Q 1/6853

(52) U.S. Cl. CPC C12Q 1/6853

(57) ABSTRACT

In some examples, a dendritic molecule may include a dendritic core; a seeding primer coupled to the dendritic core; and a plurality of dendrons, each of the dendrons comprising an inert, elongated polymer comprising a first end coupled to the dendritic core and a second end coupled to a first functional group, wherein the first functional group is to react with a second functional group to form a covalent bond. In other examples, a dendritic molecule may include a dendritic core; a single-stranded polynucleotide covalently bonded to the dendritic core; and a plurality of dendrons, each of the dendrons comprising an inert, elongated polymer comprising a first end coupled to the dendritic core and a second end.

### CROSS-REFERENCE TO RELATED APPLICATIONS

This application claims the benefit of U.S. Provisional Patent Application No. 63/585,544, filed Sep. 26, 2023 and entitled “Capturing and Amplifying Polynucleotides Using Dendritic Molecules,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2628-US.xml”, was created on Nov. 22, 2024 and is 32 kB in size.

### FIELD

This application generally relates to capturing and amplifying polynucleotides.

### BACKGROUND

Cluster amplification is an approach to amplifying polynucleotides, for example for use in genetic sequencing. Target polynucleotides are captured by primers (e.g., P5 and P7 primers) coupled to a substrate surface in a flow cell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to capturing and amplifying polynucleotides using dendritic molecules. Methods for preparing dendritic molecules also are disclosed.

Some examples herein provide a dendritic molecule for use in capturing a polynucleotide. The dendritic molecule may include a dendritic core. The dendritic molecule may include a seeding primer coupled to the dendritic core. The dendritic molecule may include a plurality of dendrons. Each of the dendrons may include an inert, elongated polymer that includes a first end coupled to the dendritic core and a second end coupled to a first functional group. The first functional group is to react with a second functional group to form a covalent bond.

Some examples further include a polynucleotide including a seeding adapter, wherein the seeding adapter is hybridized to the seeding primer. In some examples, the polynucleotide is double-stranded, and the seeding adapter is single-stranded.

In some examples, the dendritic core includes a branched polypeptide, polyester, ester, amide, ethylene glycol, or oligonucleotide.

In some examples, the inert, elongated polymer includes a polypeptide, polyester, polyamide, poly(ethylene glycol), or oligonucleotide.

In some examples, the first functional end group includes an alkyne.

Some examples herein provide a device that includes a flowcell including a plurality of wells, each well including a plurality of first functional groups. The device also may include a plurality of dendritic molecules. Each dendritic molecule may include a dendritic core; a seeding primer coupled to the dendritic core; and a plurality of dendrons. Each of the dendrons may include an inert, elongated polymer including a first end coupled to the dendritic core and a second end coupled to a second functional group. The second functional group is to react with one of the first functional groups to form a covalent bond. At least some of the wells contain a single one of the dendritic molecules.

In some examples, the first functional groups form covalent bonds with respective ones of the second functional groups. In some examples, the covalent bonds immobilize the dendritic molecule within a respective well.

In some examples, each well further includes a hydrogel to which the plurality of first functional groups is coupled

Some examples further include a polynucleotide including a seeding adapter hybridized to the seeding primer.

In some examples, the polynucleotide is double-stranded, and the seeding adapter is single-stranded.

In some examples, each well further includes a plurality of amplification primers. In some examples, the amplification primers have different sequences than the seeding primer.

In some examples, the device further includes, within wells in which a respective one of the dendritic molecules is disposed and for which the seeding adapter of one of the polynucleotides is hybridized to the seeding primer of that dendritic molecule, a substantially monoclonal cluster of amplicons.

In some examples, each of the dendritic molecules contained within a well has a hydrodynamic diameter which is about 60% to about 100% of a diameter of that well.

Some examples herein provide a method of capturing a polynucleotide in a flowcell. The method may include flowing a plurality of dendritic molecules into a flowcell including a plurality of wells. Each well may include a plurality of first functional groups. Each dendritic molecule of the plurality may include a dendritic core; a seeding primer coupled to the dendritic core; and a plurality of dendrons. Each of the dendrons may include an elongated polymer including a first end coupled to the dendritic core and a second end including a second functional group. The method may include, within at least some of the wells, respectively disposing a respective one of the dendritic molecules within that well. The method may include, for each well in which a respective one of the dendritic molecules is disposed, forming covalent bonds between the first functional groups of that well and the second functional groups of that dendritic molecule. The method may include flowing a plurality of polynucleotides into the flowcell, the polynucleotides respectively including seeding adapters. The method may include, for each well in which a respective one of the dendritic molecules is disposed, hybridizing a seeding adapter of one of the polynucleotides to the seeding primer of that dendritic molecule.

In some examples, the covalent bonds immobilize the dendritic molecule within that well.

In some examples, each well further includes a hydrogel to which the plurality of first functional groups is coupled.

In some examples, each well further includes a plurality of amplification primers. In some examples, the amplification primers have different sequences than the seeding primer. In some examples, the method further includes, for each well in which a respective one of the dendritic molecules is disposed and for which the seeding adapter of one of the polynucleotides is hybridized to the seeding primer of that dendritic molecule, using the amplification primers to generate a substantially monoclonal cluster of amplicons of the polynucleotide hybridized to the seeding primer of that dendritic molecule.

In some examples, each of the dendritic molecules may have a hydrodynamic diameter which is about 60% to about 100% of a diameter of the wells.

Some examples herein provide a dendritic molecule for use in amplifying a polynucleotide. The dendritic molecule may include a dendritic core; a single-stranded polynucleotide covalently bonded to the dendritic core; and a plurality of dendrons, each of the dendrons including an inert, elongated polymer including a first end coupled to the dendritic core and a second end.

In some examples, the dendritic core includes a branched polypeptide, polyester, ester, amide, ethylene glycol, or oligonucleotide.

In some examples, the inert, elongated polymer includes a polypeptide, polyester, polyamide, poly(ethylene glycol), or oligonucleotide.

Some examples herein provide a device. The device may include a flowcell including a plurality of wells, each well including a plurality of amplification primers. The device may include a plurality of dendritic molecules. Each dendritic molecule may include a dendritic core; a single-stranded polynucleotide covalently bonded to the dendritic core; and a plurality of dendrons. Each of the dendrons may include an inert, elongated polymer including a first end coupled to the dendritic core and a second end. At least some of the wells contain a single one of the dendritic molecules.

In some examples, each well further includes a hydrogel to which the plurality of amplification primers is coupled.

In some examples, the polynucleotide includes an amplification adapter that is hybridized to one of the amplification primers.

In some examples, each of the dendritic molecules has a hydrodynamic diameter which is about 60% to about 100% of a diameter of that well.

In some examples, the dendritic core includes a branched polypeptide, polyester, ester, amide, ethylene glycol, or oligonucleotide.

In some examples, the elongated polymer includes a polypeptide, polyester, polyamide, poly(ethylene glycol), or oligonucleotide.

Some examples herein provide a method of capturing a polynucleotide in a flowcell. The method may include flowing a plurality of dendritic molecules into a flowcell including a plurality of wells. Each well may include a plurality of amplification primers. Each dendritic molecule of the plurality may include a dendritic core; a single-stranded polynucleotide covalently bonded to the dendritic core; and a plurality of dendrons. Each of the dendrons may include an inert, elongated polymer including a first end coupled to the dendritic core and a second end. The method may include, within at least some of the wells, respectively disposing a respective one of the dendritic molecules within that well.

Some examples further include covalently bonding the single-stranded polynucleotide to the dendritic core.

In some examples, covalently bonding the single-stranded polynucleotide to the dendritic core includes: contacting a precursor of the dendritic molecule with a template polynucleotide, wherein the precursor of the dendritic molecule includes a capture primer covalently coupled to the dendritic core, and wherein the template polynucleotide includes an adapter; hybridizing the adapter to the capture primer; extending the capture primer using the template polynucleotide to form a duplex; and dehybridizing the template polynucleotide from the duplex to leave the single-stranded polynucleotide covalently coupled to the dendritic core.

Some examples further include, within wells containing a single one of the dendritic molecules, using the amplification primers to generate a substantially monoclonal cluster of amplicons of the single-stranded polynucleotide covalently coupled to the dendritic core of that dendritic molecule.

In some examples, each of the dendritic molecules contained within a well has a hydrodynamic diameter which is about 60% to about 100% of a diameter of that well.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate example operations for capturing a polynucleotide using a dendritic molecule.

FIGS. 2A-2F schematically illustrate example operations for capturing and amplifying a polynucleotide using the dendritic molecule of FIG. 1A.

FIG. 3 schematically illustrates an alternative example operation for use in capturing and amplifying a polynucleotide in the operations described with reference to FIGS. 2A-2F.

FIG. 4 schematically illustrates a device including a plurality of wells in which operations such as described with reference to FIGS. 2A-2F and/or 3 are implemented.

FIGS. 5A-5D schematically illustrate alternative example operations for capturing a polynucleotide using a dendritic molecule.

FIGS. 6A-6F schematically illustrate alternative example operations for capturing and amplifying a polynucleotide using the dendritic molecule of FIG. 5D.

FIG. 7 schematically illustrates a device including a plurality of wells in which operations such as described with reference to FIGS. 6A-6F are implemented.

FIGS. 8A-8B schematically illustrate example methods of preparing a dendritic molecule.

FIGS. 9A-9F schematically illustrate example dendritic cores that may be used in the example methods of FIGS. 8A-8B.

FIGS. 10A-10I schematically illustrate additional example dendritic cores that may be used in the example methods of FIGS. 8A-8B.

FIGS. 11A-11C schematically illustrate additional example dendritic cores that may be used in the example methods of FIGS. 8A-8B.

FIG. 12 schematically illustrates a method that was used to prepare dendritic molecules.

FIG. 13 illustrates a dynamic light scattering (DLS) trace from the dendritic molecules prepared in accordance with FIG. 12.

FIG. 14A illustrates reactions in a method with which a functionalized polymer was prepared.

FIG. 14B is a photograph of the functionalized polymer prepared in accordance with FIG. 14A.

FIG. 14C is a nuclear magnetic resonance (NMR) trace of the functionalized polymer prepared in accordance with FIG. 14A.

FIG. 14D is a gel permeation chromatography (GPC) trace of the functionalized polymer prepared in accordance with FIG. 14A, in water.

FIG. 15 schematically illustrates the way a dendritic particle prepared in accordance with FIG. 12 was used to capture and amplify a polynucleotide.

FIGS. 16A-16D are plots illustrating sequencing data obtained using the amplified polynucleotide of FIG. 15.

FIGS. 17A-17C are plots illustrating additional sequencing data obtained using the amplified polynucleotide of FIG. 15.

FIGS. 18A-18D are plots illustrating additional sequencing data obtained using the amplified polynucleotide of FIG. 15.

### DETAILED DESCRIPTION

Examples provided herein are related to capturing and amplifying polynucleotides using dendritic molecules. Methods for preparing dendritic molecules also are disclosed.

Monoclonality of a cluster in a given region of a sequencing flow cell is important to obtaining a sufficient signal to noise ratio (SNR) from that region to make a base call. The dendritic molecules herein may be used to generate substantially monoclonal clusters through a deterministic approach. More specifically, each of the dendritic molecules may include a single primer for use in capturing a single target polynucleotide via hybridization. Before or after capturing the target polynucleotide, each of the dendritic molecules may be inserted into a respective well of a flow cell. The dendritic molecules respectively may contain a plurality of dendrons which together provide the dendritic molecule with a hydrodynamic diameter which is similar to the size of the well into which the dendritic molecule is inserted. Accordingly, once inserted into the well, the dendritic molecule may sterically exclude additional molecules from being inserted into the well. Accordingly, among a plurality of wells having such dendritic molecules therein, at least some of the wells (and potentially most, if not substantially all, of the wells) respectively may contain a single dendritic molecule, and therefore may contain a single captured target polynucleotide. The target polynucleotide then may be amplified within the respective well to generate a substantially monoclonal cluster within that well.

First, some terms used herein will be briefly explained. Then, some example structures and example methods for capturing and amplifying polynucleotides using dendritic molecules, and methods of preparing dendritic molecules, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to the substrate and may hybridize to an adapter of the target polynucleotide. In some cases, a capture primer that is coupled to the substrate and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the substrate, but that may not be used to grow a complementary strand during an amplification process, may in some cases be referred to as a “seeding primer.” A capture primer that may be used to grow a complementary strand during an amplification process may in some cases be referred to as an “amplification primer.”

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM). The process creates hydrogel regions used for sequencing that may be stable over sequencing runs with a large number of cycles. The covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. Primers may be attached to hydrogel material. A solution including a plurality of target polynucleotides (e.g., a fragmented human genome or portion thereof) may then be contacted with the polished substrate such that individual target polynucleotides will seed individual wells via interactions with primers attached to the hydrogel material; however, the target polynucleotides will not occupy the interstitial regions due to absence or inactivity of the hydrogel material. Amplification of the target polynucleotides may be confined to the wells because absence or inactivity of hydrogel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, Calif.).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides) that are at different regions of an array may be differentiated from each other according to the locations of the regions in the array. An individual region of an array may include one or more molecules of a particular type. For example, a substrate region may include a single target polynucleotide having a particular sequence, or a substrate region may include several polynucleotides having the same sequence (or complementary sequences thereof). The regions of an array respectively may include different features than one another on the same substrate. Exemplary features include without limitation, wells in a substrate, beads (or other particles) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or hydrogel. Exemplary arrays in which separate substrates are located on a surface include, without limitation, those having beads in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide.

As used herein, a “dendritic molecule” is intended to refer to a molecule in which at least some of the atoms are arranged in multiple branches, or “dendrons,” which extend from a central region, or “dendritic core” (which may be referred to herein as a “core” for simplicity). A molecule including such features may be understood to be “dendritic,” even if not specifically referred to as a “dendritic molecule.” The core of a dendritic molecule may be branched. In some examples, a core of a dendritic molecule may include a “dendritic polyamide” which is intended to refer to a branched structure including amide bonds and to which elongated polymers may be coupled so as to form dendrons. A nonlimiting example of a dendritic polyamide is a “dendritic polypeptide,” which is intended to refer to a branched polypeptide to which elongated polymers may be coupled so as to form dendrons. In some examples, a capture primer (such as a seeding primer or amplification primer) may be coupled to the dendritic core and may be used to capture a polynucleotide having an adapter which is substantially complementary to the capture primer of the dendritic molecule.

As used herein, the “hydrodynamic diameter” (also known as Stokes radius) of a dendritic molecule is intended to refer to a measure of the effective size of a dendritic molecule in a fluid. It is a measure of the diameter of the sphere surrounding the dendritic molecule, based on the assumption that the dendritic molecule has the same translational diffusion coefficient as a particle. Hydrodynamic diameter is often calculated from the translational diffusion coefficient by using Stokes-Einstein equation.

### Structures and Methods for Capturing and Amplifying Polynucleotides Using Dendritic Molecules

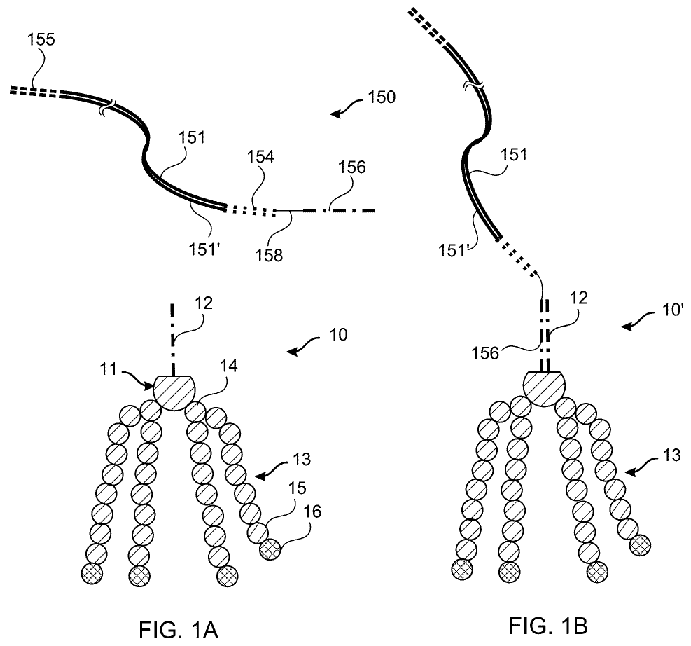

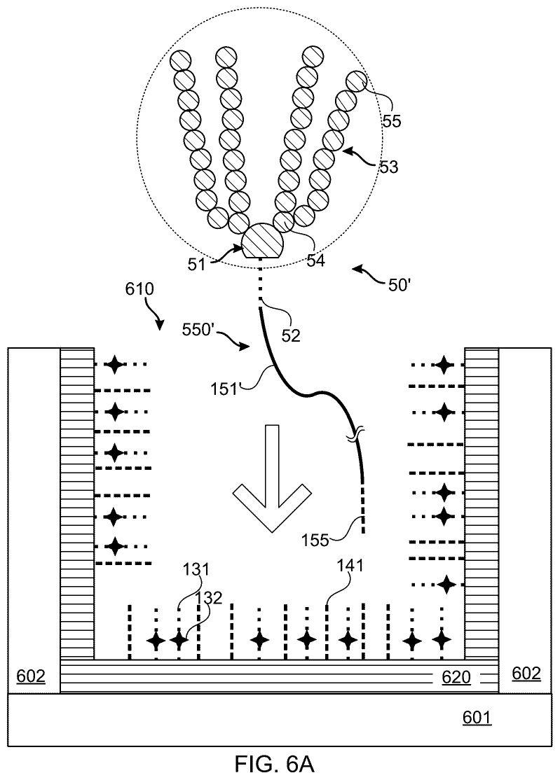

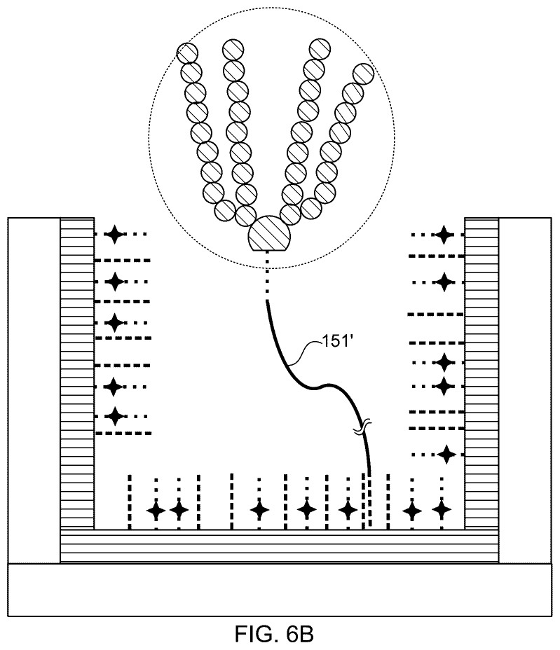

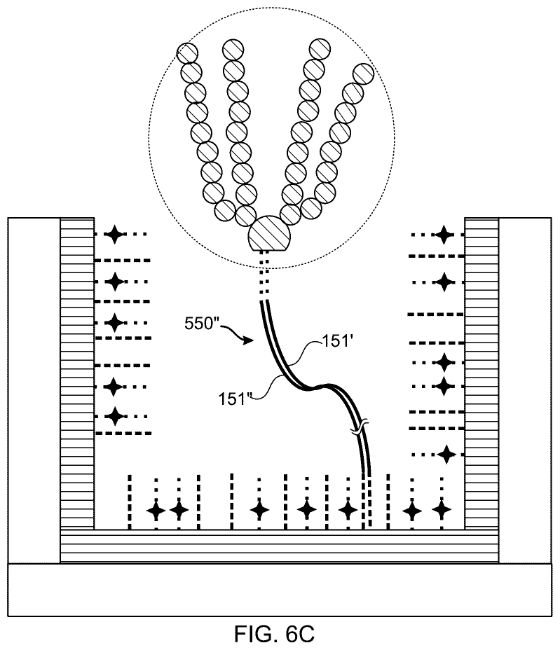

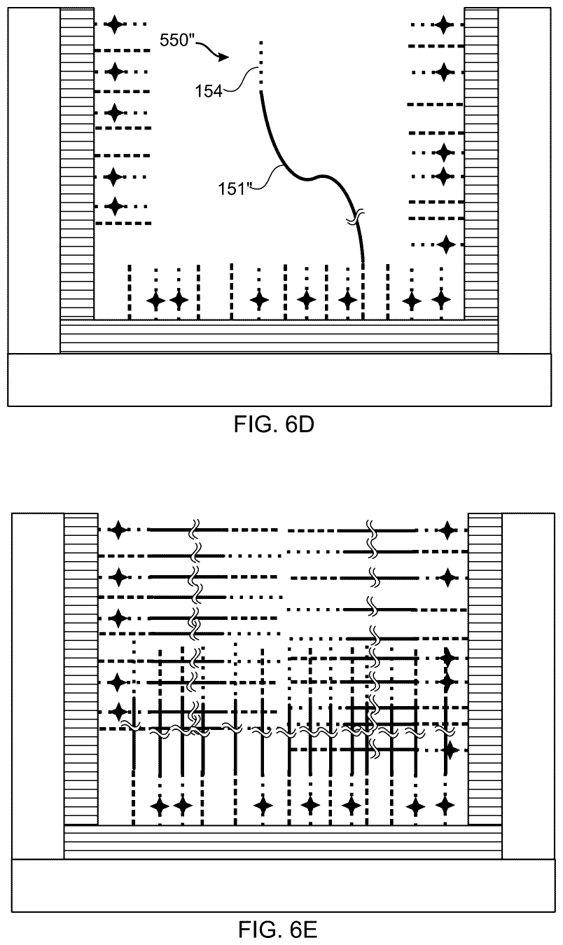

The present dendritic molecules may be used to facilitate generation of substantially monoclonal clusters. Among other things, each of the present molecules may include a single capture primer that may be used to capture a single target polynucleotide (e.g., a single single-stranded polynucleotide, or a single double-stranded polynucleotide). Additionally, each of the present dendritic molecules may have a hydrodynamic diameter which substantially matches the size of a well into which the dendritic molecule respectively becomes disposed, such that the dendritic molecule sterically inhibits any other molecule from being disposed within that well. As such, the single captured target polynucleotide may be amplified within the well so as to generate a substantially monoclonal cluster. In certain examples which will now be described with reference to FIGS. 1A-5, the dendrons of the dendritic molecule may include functional groups that can be used to covalently couple the dendritic molecule within the well, and the polynucleotide may be hybridized to the capture primer before or after the dendritic molecule is coupled to the well; such examples may be referred to as “body first” because of the manner in which the dendrons (or “body”) are inserted into, and held within, the well. Other examples, which need not necessarily include such functional groups, will be described further below with reference to FIGS. 6A-9.

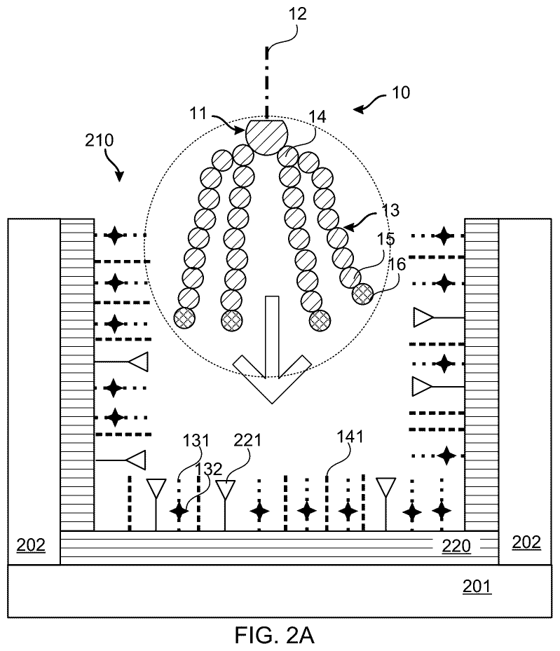

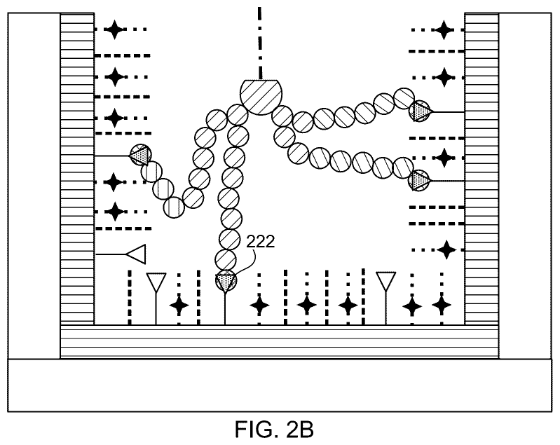

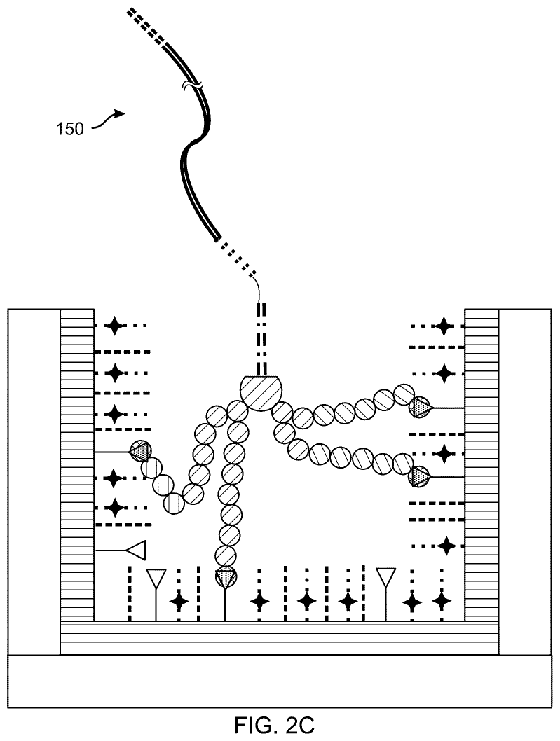

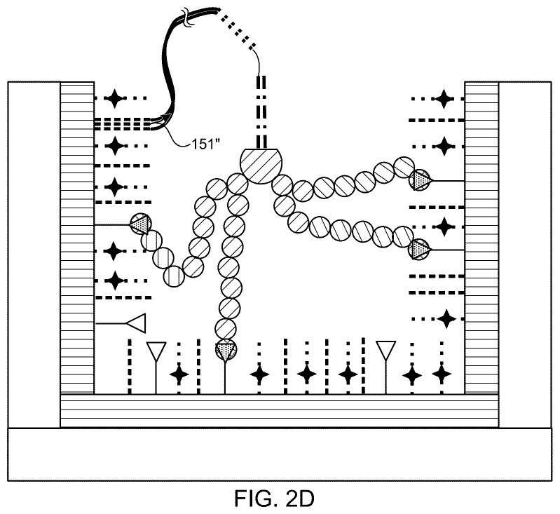

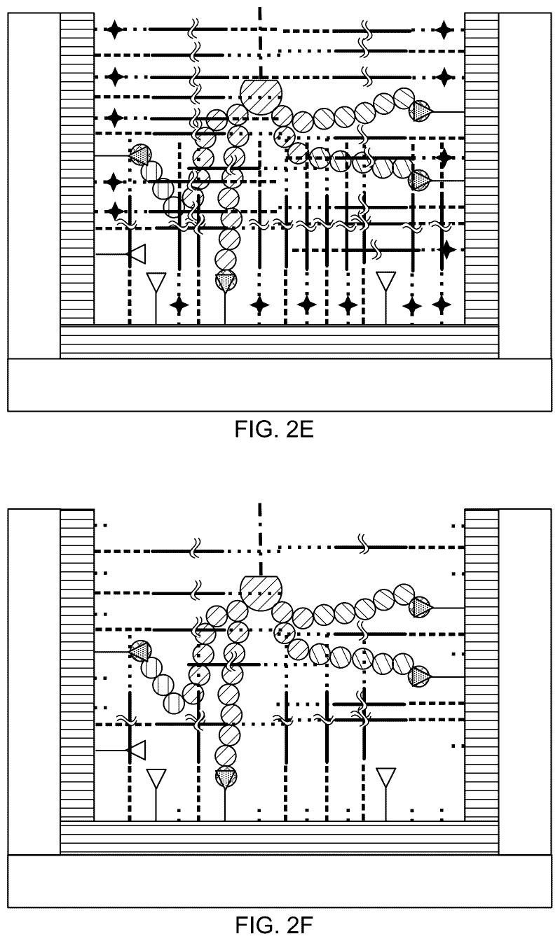

FIGS. 1A-1B schematically illustrate example operations for capturing a polynucleotide using a dendritic molecule. Referring first to FIG. 1A, dendritic molecule 10 may include dendritic core 11; seeding primer 12 coupled to the dendritic core; and a plurality of dendrons 13. Each of dendrons 13 includes an inert, elongated polymer that includes a first end 14 coupled to the dendritic core 11 and a second end 15 coupled to a functional group 16. The functional 16 group is to react with another functional group to form a covalent bond, e.g., in a manner such as will be described with reference to FIGS. 2A-2F.

As illustrated in FIG. 1A, dendritic molecule 10 may be contacted with a fluid which may include target polynucleotides having a variety of lengths, e.g., polynucleotide fragments that are generated using commercially available fragmentation or tagmentation techniques, using DNA or RNA that it is desired to sequence. Illustratively, the dendritic molecule may be contacted with a fluid including polynucleotide 150. Although only one polynucleotide is illustrated in FIG. 1A, it will be appreciated that the fluid may include thousands, or even millions, of fragments. The polynucleotides within the fluid may be substantially double-stranded. For example, polynucleotide 150 may include strand 151 which is hybridized to substantially complementary strand 151′.

Strand 151 of polynucleotide 150 may include first and second adapters, e.g., first adapter 154 and second adapter 155 which may be used in a manner such as described with reference to FIGS. 2D-2F, and complementary strand 151′ may include adapter sequences that are complementary to 154 and 155, respectively; as such, these polynucleotides and their adapters may be referred to herein as being “double-stranded.” Additionally, in the example illustrated in FIG. 1A, polynucleotide 150 optionally may further include one or more additional adapters, e.g., seeding adapter 156 which may be coupled to adapter 154 and/or adapter 155, for example via a respective linker 158. However, note that while seeding adapter 156 may be coupled to strand 151, the complementary strand 151′ may not be coupled to the complements of such seeding adapter. Accordingly, the seeding adapter 156 may be single-stranded, and thus available to hybridize with a substantially complementary seeding primer 12 in a manner such as will be described with reference to FIG. 1B. In comparison, adapters 154 and 155 are double-stranded and thus unavailable to hybridize with amplification primers on the substrate until after certain processing steps are performed, as will be explained further below with reference to FIGS. 2A-2F. While the polynucleotide 150 (and adapters 154, 155 thereof) are illustrated as being double-stranded in FIGS. 1A-1B, in other examples herein the polynucleotides may be single-stranded, and their adapters may be single stranded, depending on the example.

In some examples, in a manner such as illustrated in FIG. 1B, molecule 10 may be modified to generate modified molecule 10′ including polynucleotide 150. More specifically, seeding adapter 156 of polynucleotide 150 may be hybridized to the seeding primer 12 of molecule 10, to form modified molecule 10′. Optionally, seeding primer 12 may have the sequence PX, and seeding adapter 156 may have the sequence cPX. However, it will be appreciated that seeding primer 12 and seeding adapter 156 may have any suitable sequences which are sufficiently complementary to one another to hybridize to one another. Such hybridization may be performed while dendritic molecule 10 is in solution, e.g., suspended in the same fluid as is polynucleotide 150. Alternatively, such hybridization may be performed while dendritic molecule 10 is disposed in a well of a flowcell, e.g., in a manner which now will be described with reference to FIGS. 2A-2F.

FIGS. 2A-2F schematically illustrate example operations for capturing and amplifying a polynucleotide using the dendritic molecule 10 of FIG. 1A. A plurality of dendritic molecules 10 may be flowed into a flowcell that includes a plurality of wells 210, each well including a plurality of first functional groups 221. For example, as illustrated in FIG. 2A, the dendritic molecule 10 which is illustrated may be one of a plurality of dendritic molecules that are suspended in a fluid. In a manner such as described with reference to FIG. 1A, each dendritic molecule 10 of the plurality may include a dendritic core 11; a seeding primer 12 coupled to the dendritic core; and a plurality of dendrons 12, each of the dendrons including an elongated polymer including a first end 14 coupled to the dendritic core and a second end 15 including a second functional group 16.

The fluid containing the dendritic molecules 10 may be flowed into a flowcell that includes well 210 which is one of a plurality of wells within substrate 200. Wells 210 may include a volume which is bounded, for example, by a bottom substrate 201 and one or more vertical sidewalls 202. The volume may be generated in any suitable manner, for example using nanoimprint lithography, conventional photolithography techniques, or the like. In the nonlimiting example illustrated in FIG. 2A, a hydrogel 220 may be disposed within respective wells 210, and may be coupled to first functional groups 221. Additionally, hydrogel 221 may be coupled to a mixture of amplification primers 131 and amplification primers 141. Amplification primers 131 and 141 may have any suitable sequences, e.g., that are orthogonal to one another. That is, amplification primer 141 may be referred to as an orthogonal amplification primer, while amplification primer 131 may be referred to as an amplification primer; or amplification primer 131 may be referred to as an orthogonal amplification primer, while amplification primer 141 may be referred to as an amplification primer. In one nonlimiting example, amplification primers 131 may include P5, and amplification primers 141 may include P7. In another nonlimiting example, amplification primers 141 may include P5, and amplification primers 131 may include P7.

Within at least some of the wells 210, a respective one of the dendritic molecules 10 may become disposed within that well. For example, as intended to be represented in FIG. 2A by the large downward-pointing arrow, dendritic molecule 10 may become inserted into well 210. For example, diffusion may transport the dendritic molecule 10 to well 210, and then interactions between the dendritic molecule and hydrogel 220 within the well may retain the dendritic molecule within the well. In various examples, the dendrons of dendritic molecule 10 may covalently, non-covalently, and/or electrostatically interact with the polymer of hydrogel 220. Additionally, as intended to be illustrated within FIG. 2A, dendritic molecule 10 may have a hydrodynamic diameter denoted by the dotted circle, which sterically inhibits other dendritic molecules from entering the same well 210. As such, well 210 (and other wells within the flowcell) may receive substantially a single one of the dendritic molecules 10. For example, each of the dendritic molecules 10 may have a hydrodynamic diameter which is about 60% to about 100% of a diameter of wells 210.

As illustrated in FIG. 2B, within wells 210 containing a single one of the dendritic molecules, covalent bonds 222 are formed between the first functional groups 221 of that well and the second functional groups 16 of that molecule. In FIG. 2B, covalent bonds 222 are intended to be represented by the shading of the triangles representing first functional groups 221, indicating that the first functional groups have reacted. The covalent bonds may immobilize the dendritic molecule 10 within well 210, such that the dendritic molecule may remain in the well during subsequent capture and clustering operations which now will be described. As such, the captured polynucleotide may be retained within that well, reducing the risk that the polynucleotide inadvertently may seed a different well.

In some examples, after the dendritic molecule 10 becomes coupled within the well 210, plurality of polynucleotides are flowed into the flowcell. The polynucleotides may be configured substantially as described with reference to FIGS. 1A-1B, e.g., respectively may include seeding adapters 156 that can hybridize to (e.g., are complementary to) seeding primers 12 of dendritic particles 10. As illustrated in FIG. 2C, for each well in which a respective one of the dendritic molecules is disposed, a seeding adapter 156 of one of the polynucleotides 150 may hybridize to the seeding primer 12 of that molecule. In some examples, the amplification primers 131, 141 within wells 210 may have different sequences than the seeding primers 12 of dendritic molecules 10, e.g., may be orthogonal to the seeding primers. As such, although polynucleotides 150 may come into contact with amplification primers 131, 141 while being flowed through the flowcell, seeding adapters 156 may be substantially unable to hybridize to such amplification primers because they are not complementary. Additionally, during operations such as illustrated in FIG. 2C, seeding adapters 154, 155 of polynucleotides 150 may be substantially unable to hybridize to amplification primers 131, 141 because they are double-stranded. Accordingly, capture of polynucleotides 150 are substantially limited to that by seeding primers 12. Additionally, because a single seeding primer is expected to be within each well, because a single dendritic molecule can fit within each well, a single polynucleotide 150 is expected to be captured at each well.

For each well in which a respective one of the dendritic molecules is disposed and for which the seeding adapter of one of the polynucleotides is hybridized to the seeding primer of that dendritic molecule, the amplification primers 131, 141 may be used to generate a substantially monoclonal cluster of amplicons of the polynucleotide hybridized to the seeding primer of that molecule. For example, as illustrated in FIG. 2D, double-stranded adapter 155 of captured polynucleotide 150 hybridizes with one of primers 141 to form a triplex using a process that may be referred to as “strand invasion” and may be promoted using a recombinase (not specifically illustrated in FIG. 2D). An amplified cluster then may be formed for the polynucleotide which is captured within each respective well 210. For example, FIG. 2D illustrates recombinase-promoted extension of the primer 141 to which double-stranded adapter 155 hybridizes to form amplicon 151″ which is covalently coupled to hydrogel 220. Amplicon 151″ repeatedly may be further amplified using strand invasion. For example, it may be seen that the composition of FIG. 2E includes a plurality of amplicons that are formed using a mixture of primers 131 and 141. The amplicons may extend linearly away from the substrate as illustrated in FIG. 2E. In the example illustrated in FIG. 2E, capture primers 131 may include excision moieties 132 which may be used to remove amplicons that are oriented in a selected direction, so that the remaining amplicons are oriented substantially in the same direction as one another in a manner such as illustrated in FIG. 2F. In some examples, in wells 210 within substantially a single polynucleotide 150 is captured using a respective dendritic molecule 10 and amplified using the amplification primers within that well, the cluster of amplicons within that respective well 210 may be expected to be substantially monoclonal. Note that during amplification, the original polynucleotide 150 which was captured optionally may become dehybridized from seeding primer 12.

For further details regarding seeding and amplification operations using strand invasion and seeding adapters and seeding primers that are orthogonal to capture adapters and capture primers, see International Patent Application No. PCT/US2022/053002, now International Patent Publication No. WO2023/114394, filed on Dec. 15, 2022 and entitled “Orthogonal Hybridization,” the entire contents of which are incorporated by reference herein.

As noted above with reference to FIG. 1A, linker 158 may be provided between adapter 154 and seeding adapter 156. In some examples, linker 158 may be or include one or more of: a carbon-containing chain with a formula (CH₂)ₙ wherein “n” is from 1 to about 1500, for example less than about 1000, preferably less than 100, e.g. from 2-50, particularly 5-25; polyethylene glycol (PEG); or iSp9 (Spacer 9) which is a triethylene glycol spacer that can be incorporated at the 5′-end or 3′-end of an oligo or internally. Linkers formed primarily from chains of carbon atoms or from PEG may be modified so as to contain functional groups which interrupt the chains. Examples of such groups include ketones, esters, amines, amides, ethers, thioethers, sulfoxides, sulfones. Separately or in combination with the presence of such functional groups, groups such as alkene, alkyne, aromatic or heteroaromatic moieties, or cyclic aliphatic moieties (e.g., cyclohexyl) may be included. Cyclohexyl or phenyl rings may, for example, be connected to a PEG or (CH₂)ₙ chain through their 1- and 4-positions. Other linkers are envisaged which are based on nucleic acids or monosaccharide units (e.g., dextrose). It is also within the scope of this disclosure to utilise peptides as linkers. A variety of other linkers may be employed. The linker preferably is stable under conditions under which the polynucleotides are intended to be used subsequently, e.g., conditions used in DNA amplification. The linker should also be such that it is not by-passed by DNA polymerases, terminating DNA polymerization before copying the seeding adapter 156 sequence (if it is nucleotide based such as a PX′ sequence). This allows for the seeding adapter to remain single-stranded and available for hybridization at all times.

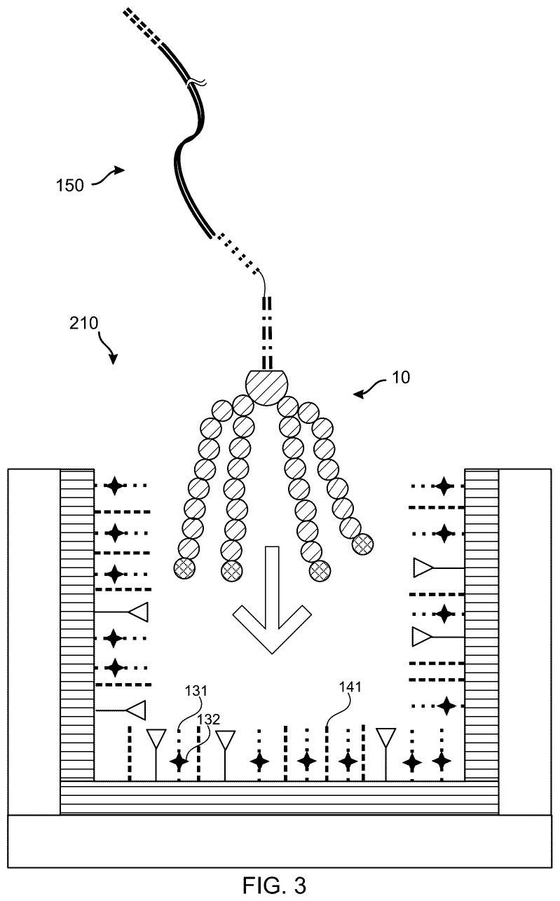

Note that FIGS. 2A-2F describe only one possible order of operations in which dendritic particles 10 may be used to capture and amplify respective polynucleotides 150. For example, FIG. 3 schematically illustrates an alternative example operation for use in capturing and amplifying a polynucleotide in the operations described with reference to FIGS. 2A-2F. In the example shown in FIG. 3, dendritic particle 10 captures polynucleotide 150 in solution in a manner such as described with reference to FIG. 1B, and the particle (with polynucleotide 150 coupled thereto) is disposed within a corresponding well 210. The first reactive groups of the well 210 then may react with the second reactive groups of the dendritic particle 10, to provide a structure such as described with reference to FIG. 2C. The captured polynucleotide 150 then may be amplified in a manner such as described with reference to FIGS. 2D-2F. Note that in these examples, seeding primer 12 need not necessarily be orthogonal to amplification primers 131 and/or 141, because polynucleotide capture is performed off of the flow cell such that seeding adapter 156 substantially may not have the opportunity to hybridize to the amplification primers.

FIG. 4 schematically illustrates a device including a plurality of wells in which operations such as described with reference to FIGS. 2A-2F and/or 3 are implemented. Device 400 includes a flowcell 410 including a plurality of wells 210 such as described with reference to FIG. 2A, each well including a plurality of first functional groups 221 (functional groups not specifically shown in FIG. 4); and a plurality of dendritic molecules 10 such as described with reference to FIG. 1A, e.g., including seeding primers 12. The dendritic molecules 10 may be suspended in a fluid which is flowed into the flowcell in a manner as intended to be suggested by the large arrow, and once disposed in a respective well the second functional groups 16 of a dendritic molecule may become covalently bonded to the first functional groups 221. As illustrated in FIG. 4, at least some of the wells (and optionally a majority of the wells, or more than 90% of the wells, or even substantially all of the wells) contain a single one of the dendritic molecules. In a manner such as described with reference to FIG. 2B, the first functional groups may form covalent bonds with respective ones of the second functional groups, and the covalent bonds may immobilize the dendritic molecule within that well. In some examples, the device may include polynucleotide(s) 150 which includes a seeding adapter hybridized to the seeding primer, e.g., as described with reference to FIGS. 2C and 3. Similarly as intended to be represented by the large arrow in FIG. 4, the polynucleotides may be flowed into the flowcell and there may hybridize to the seeding primers of respective dendritic molecules within respective wells 210. The polynucleotide(s) may be double-stranded, and the seeding adapter may be single-stranded, e.g., as described with reference to FIGS. 1A-1B. In some examples, the device further may include, within wells in which a respective one of the dendritic molecules is disposed and for which the seeding adapter of one of the polynucleotides is hybridized to the seeding primer of that dendritic molecule, a substantially monoclonal cluster of amplicons, e.g., as described with reference to FIG. 2E or 2F.

In other examples provided herein, the dendrons of the dendritic molecule may be substantially inert (that is, do not include functional groups that can be used to covalently couple the dendritic molecule within the well). Instead, the polynucleotide is hybridized to the capture primer in solution, and the capture primer then extended to form a single amplicon which is covalently coupled to the dendritic molecule.

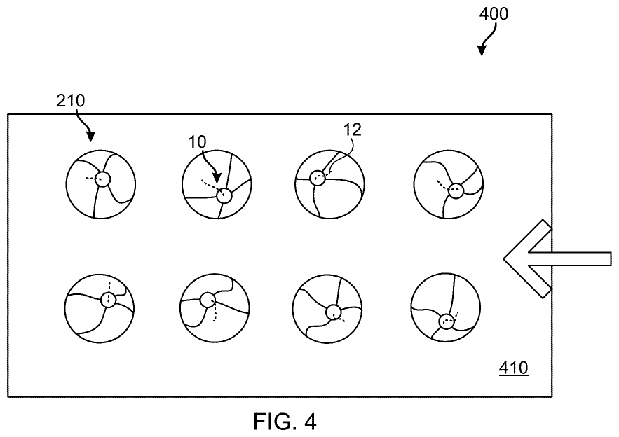

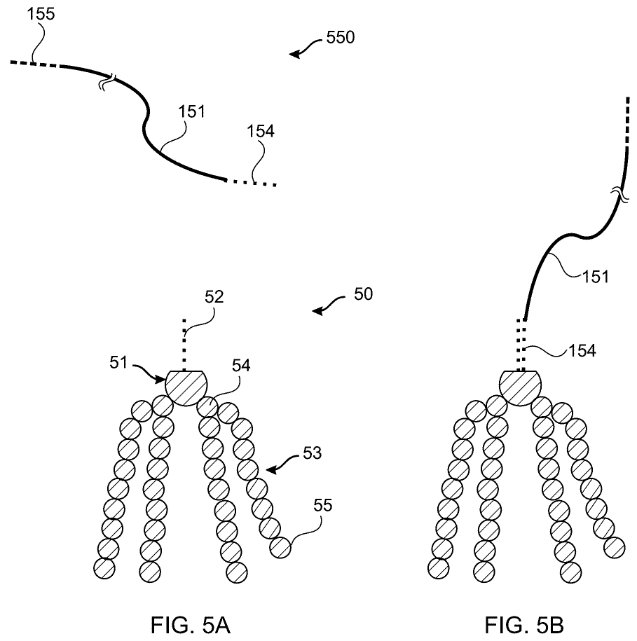

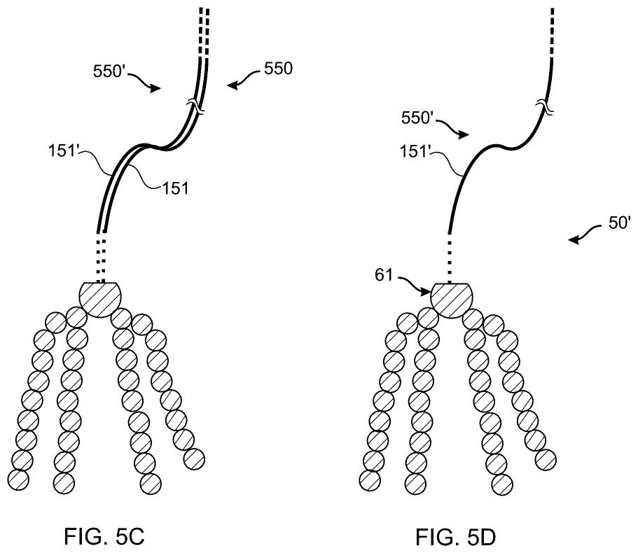

For example, FIGS. 5A-5D schematically illustrate alternative example operations for capturing a polynucleotide using a dendritic molecule. Referring first to FIG. 5A, dendritic molecule 50 may include dendritic core 51; seeding primer 52 coupled to the dendritic core; and a plurality of dendrons 53. Each of dendrons 53 includes an inert, elongated polymer that includes a first end 54 coupled to the dendritic core 51 and a second end 55 which, in the illustrated embodiment, is substantially inert. Optionally, the second end 55 may be coupled to a functional group 16 (not specifically illustrated) in a manner such as described with reference to FIG. 1A.

As illustrated in FIG. 5A, dendritic molecule 50 may be contacted with a fluid which may include target polynucleotides having a variety of lengths, e.g., polynucleotide fragments that are generated using commercially available fragmentation or tagmentation techniques, using DNA or RNA that it is desired to sequence. Illustratively, the dendritic molecule may be contacted with a fluid including polynucleotide 550. Although only one polynucleotide is illustrated in FIG. 5A, it will be appreciated that the fluid may include thousands, or even millions, of fragments. The polynucleotides within the fluid may be substantially single-stranded. Polynucleotide 550 may include first and second adapters, e.g., first adapter 154 and second adapter 155, and target strand 251. While the polynucleotide 550 (and adapters 154, 155 thereof) are illustrated as being double-stranded in FIGS. 5A-5B, in other examples herein the polynucleotides may be double-stranded, and their adapters may be double stranded, depending on the example.

In some examples, in a manner such as illustrated in FIG. 5B, adapter 154 of polynucleotide 550 may be hybridized to the seeding primer 52 of molecule 50. In some examples, seeding primer 52 may have the sequence P5 or P7, and adapter 154 may have the complementary sequence cP5 or cP7 as appropriate. However, it will be appreciated that seeding primer 52 and adapter 154 may have any suitable sequences which are sufficiently complementary to one another to hybridize to one another. Such hybridization may be performed while dendritic molecule 50 is in solution, e.g., suspended in the same fluid as is polynucleotide 550. Optionally, as illustrated in FIG. 5C, adapter 154 may be extended (e.g., using a polymerase and nucleotides, not specifically illustrated) to form an amplicon 550′ (including target polynucleotide complement 151′) of polynucleotide 550. The amplicon 550′ may be covalently bonded to dendritic molecule 50 via that adapter 154. As illustrated in FIG. 5D, the original polynucleotide 550 then may be dehybridized, leaving a single-stranded amplicon 550′ coupled to the modified dendritic molecule 50′. In a manner which will now be described with reference to FIGS. 6A-6F, amplicon 550′ may be amplified in such a manner as to generate a substantially monoclonal cluster.

FIGS. 6A-6F schematically illustrate example operations for capturing and amplifying a polynucleotide using the dendritic molecule 50′ of FIG. 5D. A plurality of dendritic molecules 50′ may be flowed into a flowcell that includes a plurality of wells 610. For example, as illustrated in FIG. 6A, the dendritic molecule 50 which is illustrated may be one of a plurality of dendritic molecules that are suspended in a fluid. In a manner such as described with reference to FIG. 5D, each dendritic molecule 50′ of the plurality may include a dendritic core 51; a seeding primer 52 coupled to the dendritic core; and a plurality of dendrons 52, each of the dendrons including an elongated polymer including a first end 54 coupled to the dendritic core and a second end 55. Additionally, the dendritic molecule 50′ may include a single-stranded polynucleotide covalently bonded to dendritic core 51, e.g., via seeding primer 52.

The fluid containing the dendritic molecules 50′ may be flowed into a flowcell that includes well 610 which is one of a plurality of wells within substrate 600. Wells 610 may include a volume which is bounded, for example, by a bottom substrate 601 and one or more vertical sidewalls 602. The volume may be generated in any suitable manner, for example using nanoimprint lithography, conventional photolithography techniques, or the like. In the nonlimiting example illustrated in FIG. 6A, a hydrogel 620 may be disposed within respective wells 610, and may be coupled to a mixture of amplification primers 131 and amplification primers 141 which may be configured in the manner described with reference to FIG. 2A.

Within at least some of the wells 610, a respective one of the dendritic molecules 50′ may become disposed within that well. For example, as intended to be represented in FIG. 6A by the large downward-pointing arrow, dendritic molecule 50′ may become inserted into well 610, e.g., as a result of diffusion. Additionally, as intended to be illustrated within FIG. 6A, dendritic molecule 50′ may have a hydrodynamic diameter denoted by the dotted circle, which sterically inhibits other dendritic molecules from entering the same well 610. As such, well 610 (and other wells within the flowcell) may receive substantially a single one of the dendritic molecules 50′. For example, each of the dendritic molecules 50′ may have a hydrodynamic diameter which is about 60% to about 100% of a diameter of wells 610.

As illustrated in FIG. 6B, the available adapter 155 of polynucleotide 550′ may hybridize to an amplification primer 141 within well 610, thus temporarily binding dendritic molecule 50′ within that well. In this regard, note that dendritic molecule 50′ may be considered to have a “head-first” orientation while bound within well 610 illustrated in FIG. 6B, while dendritic molecule 10 may be considered to have a “body-first” orientation while bound within well 210 illustrated in FIG. 2B. Additionally, while covalent bonds may be formed to immobilize the dendritic molecule 10 within well 210 such that a polynucleotide subsequently may be coupled thereto in a manner such as described with reference to FIG. 2C, dendritic molecule 50′ may not necessarily be immobilized within well 610 in such a manner. Nonetheless, the hybridization between adapter 155 and amplification primer 141 is sufficient to retain dendritic molecule 50′ in well 610 for a sufficient amount of time to generate an amplicon of polynucleotide 550′ which is covalently coupled within the well, and that may be used during subsequent clustering operations in a manner which now will be described. As such, the amplicon of the captured polynucleotide may be retained within that well, reducing the risk that the amplicon inadvertently may seed a different well. Additionally, because a single dendritic molecule can fit within each well, a single polynucleotide 550′ is expected to be inserted into, and amplified within, each well.

In some examples, after adapter 155 is hybridized to amplification primer 141, the amplification primer 141 is extended in a manner such as illustrated in FIG. 6C (e.g., using nucleotides and a polymerase, not specifically illustrated). Such extension may generate amplicon 550″ including strand 151″ which is complementary to complementary strand 151′ and is covalently coupled within well 610 via amplification primer 141. Note that strand 151″ thus may have substantially the same sequence as strand 151 of polynucleotide 550 described with reference to FIG. 5A. Following such extension, amplicon 550′ may be dehybridized from amplicon 550″, which causes dendritic molecule 50′ to dissociate from the well. At this time, the well may be considered to have been seeded with amplicon 550″, and that amplicon then may be used to generate a cluster. For example, as illustrated in FIG. 6D, the free end of amplicon 550″ includes adapter 154 which may hybridize to an amplification primer 131 (hybridization not specifically illustrated). The amplicon 550″ then may be amplified using bridge amplification or other suitable method for generating amplicons. For example, it may be seen that the composition of FIG. 6E includes a plurality of amplicons that are formed using a mixture of primers 131 and 141. The amplicons may extend linearly away from the substrate as illustrated in FIG. 6E. In the example illustrated in FIG. 6E, capture primers 131 may include excision moieties 132 which may be used to remove amplicons that are oriented in a selected direction, so that the remaining amplicons are oriented substantially in the same direction as one another in a manner such as illustrated in FIG. 6F. In some examples, in wells 610 within substantially a single polynucleotide 550′ is captured using a respective dendritic molecule 50′ and amplified using the amplification primers within that well, the cluster of amplicons within that respective well 610 may be expected to be substantially monoclonal.

FIG. 7 schematically illustrates a device including a plurality of wells in which operations such as described with reference to FIGS. 6A-6F are implemented. Device 700 includes a flowcell 710 including a plurality of wells 610 such as described with reference to FIG. 6A; and a plurality of dendritic molecules 50′ such as described with reference to FIG. 5A, e.g., respectively including single stranded polynucleotides 550′. The dendritic molecules 50′ may be suspended in a fluid which is flowed into the flowcell in a manner as intended to be suggested by the large arrow. As illustrated in FIG. 7, at least some of the wells (and optionally a majority of the wells, or more than 90% of the wells, or even substantially all of the wells) contain a single one of the dendritic molecules. In a manner such as described with reference to FIG. 6B, the adapter of the polynucleotide 550′ may become hybridized to an amplification primer in the well and thus retain the polynucleotide within the well for sufficient time to generate an amplicon of the polynucleotide in a manner such as described with reference to FIGS. 6C-6F. In some examples, the device further may include, within wells in which a respective one of the dendritic molecules is disposed and for which the seeding adapter of one of the polynucleotides is hybridized to the seeding primer of that dendritic molecule, a substantially monoclonal cluster of amplicons, e.g., as described with reference to FIG. 6E or 6F.

Further details regarding example configurations of dendritic molecules, example materials suitable for use in dendritic molecules, and example methods of preparing dendritic molecules, now will be provided.

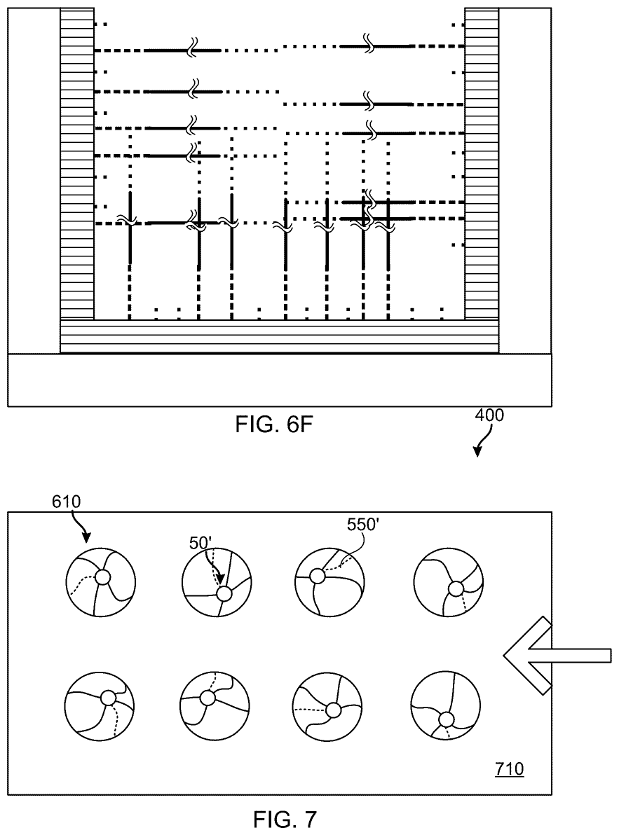

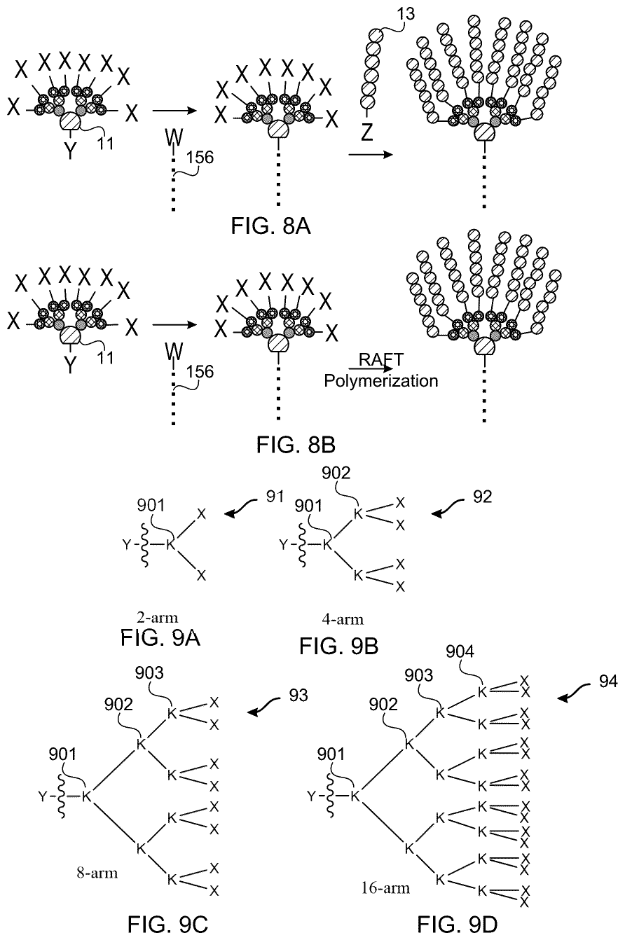

The present dendritic molecules may include any suitable number of dendrons coupled to the dendritic core. The dendrons and the seeding primer may be coupled to the dendritic core in any suitable manner. For example, FIGS. 8A-8B schematically illustrate example methods of preparing a dendritic molecule. FIG. 8A illustrates a “grafting-to” example in which dendritic core 11 includes a functional group “Y” which may be reacted with a functional group “W” which is coupled to seeding primer 156, to couple the seeding primer to the dendritic core. In this example, dendritic core also includes a functional group “X” which may be reacted with a functional group “Z” which is coupled to an elongated polymer 13, to couple the elongated polymer to the dendritic core and thus form a dendron. Optionally, the free end of the elongated polymer 13 may include a functional group (not specifically illustrated in FIG. 8A) corresponding to functional group 16 described with reference to FIGS. 1A-1B and 2A-2F.

In some examples, seeding adapter 156 and/or elongated polymer 13 illustrated in FIG. 8A respectively are attached to dendritic core 11 by covalent bonding. In some such examples, the covalent bonding is selected from the group consisting of amine-NHS ester bonding, amine-imidoester bonding, amine-pentafluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxyamine-aldehyde bonding, SpyTag-SpyCatcher bonding, Snap-tag-O⁶-benzylguanine bonding, CLIP-tag-O²-benzylcytosine bonding, and sortase-coupling bonding.

Illustratively, dendritic core 11 may include one or more amino bonding sites, carboxy bonding sites, thiol bonding sites, alkyne bonding sites, NHS bonding sites, maleimide bonding sites, hydrazide bonding sites, alkoxyamine bonding sites, tetrazine bonding sites, aldehyde bonding sites, azido bonding sites, hydroxy bonding sites, cycloalkene bonding sites (such as transcyclooctene bonding sites or norbornene bonding sites), cycloalkyne bonding sites (such as cyclooctyne bonding sites dibenzocyclooctyne (DBCO) bonding sites, or bicyclononyne bonding sites), oxyamine bonding sites, SpyTag bonding sites, Snap-tag bonding sites, CLIP-tag bonding sites, or proteins with N-terminus recognized by sortase, or combinations thereof. In some such examples, seeding adapter 156 and/or elongated polymers 13 respectively include a functional moiety that allows for covalent bonding with the corresponding site of the dendritic core. The functional moiety of the seeding adapter 156 and/or elongated polymers 13 independently may include or is selected from a NHS ester moiety, an aldehyde moiety, an imidoester moiety, a pentafluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other examples, the functional moiety on the dendritic core and the functional moiety of the seeding adapter 156 and/or elongated polymers 13 may be reversed from the specific examples provided above. Preferably, the moiety W on seeding adapter 156 is of a different type different than the moiety Z on elongated polymer 13, and the dendritic core includes different types of moieties to respectively couple to seeding adapter 156 and to elongated polymer 13.

A non-exclusive list of complementary binding partners is presented in Table 1:

TABLE 1

| | Example moiety “Y” on | Example moiety “W” on seeding |
|---|---|---|
| | dendritic core 11 or moiety | adapter 156 or moiety “Z” on |
| | “W” on seeding adapter 156 or | elongated polymer 13 or moiety “Y” |
| Bonding site | moiety “Z” on elongated polymer 13 | on dendritic core |
| amine-NHS | amine group, —NH₂ | 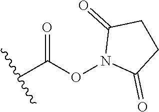 [SMILES: *C(=O)ON1C(=O)CCC1=O] |
| | | N-Hydroxysuccinimide ester |
| amine-imidoester | amine group, —NH₂ | 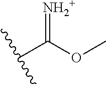 [SMILES: *C(=[NH2+])OC] |
| | | imidoester |
| amine- pentafluorophenyl ester | amine group, —NH₂ | 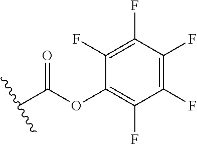 [SMILES: *C(=O)Oc1c(F)c(F)c(F)c(F)c1F] |
| | | pentafluorophenyl ester, |
| amine- hydroxymethyl phosphine | amine group, —NH₂ | 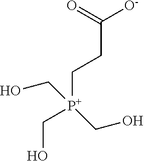 [SMILES: O=C([O-])CC[P+](CO)(CO)CO] |
| | | hydroxymethyl phosphine |
| amine-carboxylic | amine group, —NH₂ | carboxylic acid group, —C(═O)OH (e.g., |
| acid | | following activation of the carboxylic |
| | | acid by a carbodiimide such as EDC (1- |
| | | ethyl-3-(-3-dimethylaminopropyl) |
| | | carbodiimide hydrochloride) or DCC |
| | | (N′,N′-dicyclohexyl carbodiimide) to |
| | | allow for formation of an amide bond |
| | | of the activated carboxylic acid with an |
| | | amine group) |
| thiol-maleimide | thiol, —SH | 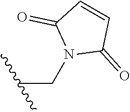 [SMILES: *CN1C(=O)C=CC1=O] |
| | | maleimide |
| thiol-haloacetyl | thiol, —SH | 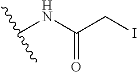 [SMILES: *NC(=O)CI] |
| | | haloacetyl (e.g., iodoacetyl or other |
| | | haloacetyl) |
| thiol-pyridyl disulfide | thiol, —SH | 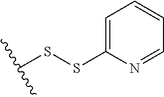 [SMILES: *SSc1ccccn1] |
| | | pyridyl disulfide |
| thiol-thiosulfonate | thiol, —SH | 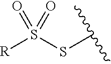 |
| | | thiosulfonate |
| thiol-vinyl sulfone | thiol, —SH | 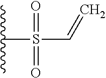 [SMILES: *S(=O)(=O)C=C] |
| | | vinyl sulfone |
| aldehyde- hydrazide | aldehyde, —C(═O)H | 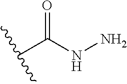 [SMILES: *C(=O)NN] |
| | | hydrazide |
| aldehyde- alkoxyamine | aldehyde, —C(═O)H | 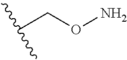 [SMILES: *CON] |
| | | alkoxyamine |
| hydroxy- isocyanate | hydroxyl, —OH | 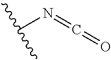 [SMILES: *N=C=O] |
| | | isocyanate |
| azide-alkyne | azide, —N₃ | 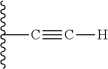 [SMILES: *C#C] |
| | | alkyne |
| azide-phosphine | azide, —N₃ | 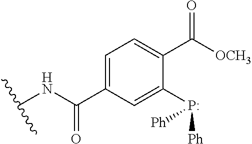 |
| | | phosphine, e.g.: |
| azide-cyclooctyne | azide, —N₃ | 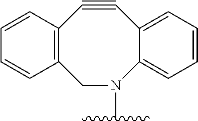 [SMILES: *N1Cc2ccccc2C#Cc2ccccc21] |
| | | cyclooctyne, e.g. dibenzocyclooctyne |
| | | (DBCO) |
| | | or |
| | | 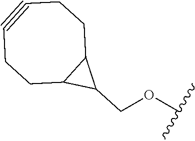 [SMILES: *OCC1C2CCC#CCCC21] |
| | | BCN (bicyclo[6.1.0]nonyne) |
| azide-norbornene | azide, —N₃ | 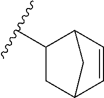 [SMILES: *C1CC2C=CC1C2] |
| | | norbornene |
| transcyclooctene- tetrazine | 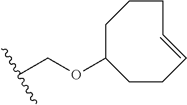 [SMILES: *COC1CC/C=C/CCC1] | 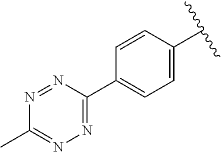 [SMILES: *c1ccc(-c2nnc(C)nn2)cc1] |
| | transcyclooctene | tetrazine, e.g., benzyl-methyltetrazine |
| norbornene- tetrazine | 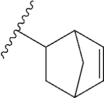 [SMILES: *C1CC2C=CC1C2] | 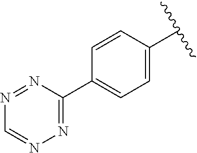 [SMILES: *c1ccc(-c2nncnn2)cc1] |
| | norbornene | tetrazine, e.g. benzyl-tetrazine |
| oxime | aldehyde or ketone (e.g., amine | alkoxyamine |
| | group or N-terminus of | |
| | polypeptide converted to an | |
| | aldehyde or ketone by pyridoxal | |
| | phosphate) | |
| SpyTag- | SpyTag: amino acid sequence | SpyCatcher amino acid sequence: |
| SpyCatcher | AHIVMVDAYKPTK (SEQ ID | MKGSSHHHHHHVDIPTTENLYFQ |
| | NO: 9) | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK (SEQ ID |
| | | NO: 10) |
| SNAP-tag-O⁶- Benzylguanine | SNAP-tag (O-6-methylguanine- DNA methyltransferase) | 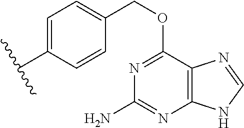 [SMILES: *c1ccc(COc2nc(N)nc3[nH]cnc23)cc1] |
| | | O⁶-Benzylguanine |
| CLIP-tag-O²- benzylcytosine | CLIP-tag (modified O-6- methylguanine-DNA methyltransferase) | 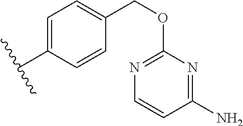 [SMILES: *c1ccc(COc2nccc(N)n2)cc1] |
| | | O²-benzylcytosine |
| Sortase-coupling | -Leu-Pro-X-Thr-Gly (SEQ ID | -Gly₍₃₋₅₎ (SEQ ID NO: 12) (SEQ ID |
| | NO: 11) | NO: 13) |

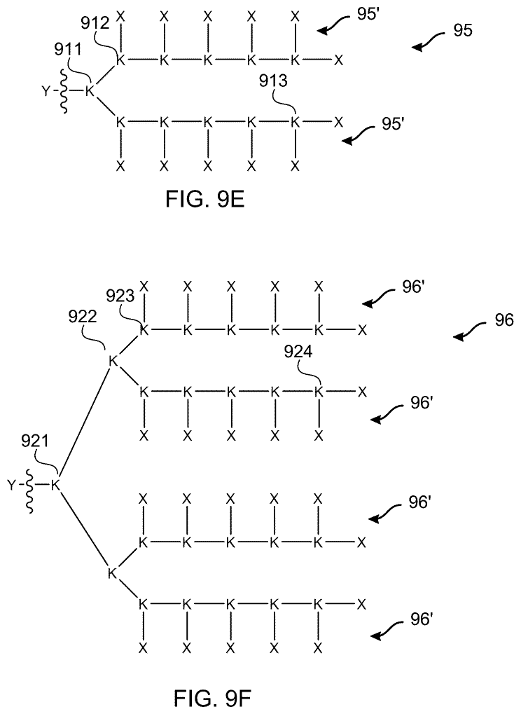

While FIG. 8A illustrates an example in which the elongated polymer is formed before being coupled to the dendritic core to form the dendrons, in other examples the elongated polymer is grown directly from the dendritic core, e.g., using RAFT polymerization or atom transfer radical polymerization (ATRP) to form the dendrons. For example, FIG. 8B illustrates an example in which the primer is coupled using a “grafting-to” approach and the dendrons are coupled using a “grafting-from” approach. More specifically, in the nonlimiting example of FIG. 8B, dendritic core 11 includes a functional group “Y” which may be reacted with a functional group “W” which is coupled to seeding primer 156, to couple the seeding primer to the dendritic core in a manner such as described with reference to FIG. 8A. In this example, dendritic core also includes a functional group “X” from which an elongated polymer 13 may be grown (e.g., using RAFT polymerization or ATRP) to form a dendron. In some examples, X is a RAFT agent (such as a dithiobenzoate, trithiocarbonate, dithiocarbamate, or xanthate) or a chain transfer agent (CTA) for RAFT, or a halogen-containing macroinitiator for ATRP. Some examples of controlled radical polymerizations that may be used to form elongated polymer 13 include, but are not limited to, RAFT, ATRP, nitroxide-mediated polymerization (NMP), catalytic chain transfer polymerization (CCTP). In examples in which the elongated polymer 13 is formed using RAFT polymerization, the polymer may include, for example, a polystyrene, polyacrylate, polyacrylamide, polymethacrylate, polymethacrylamide, polyvinyl ester, or polyvinyl amide. For further details regarding forming dendritic polymers using polymerization, see the following references, the entire contents of each of which are incorporated by reference herein: Taton et al., “Controlled polymerizations as tools for the design of star-like and dendrimer-like polymers,” Polymer International, 55(1): 1138-1145 (2006); and Gillies et al., “Dendrimers and dendritic polymers in drug delivery,” Drug Discovery Today 10(1): 35-43 (2005). In still other examples such as described with reference to FIGS. 9A-9F, elongated polymer 13 may be formed using peptide synthesis or oligosynthesis. Optionally, the free end of the elongated polymer 13 may include a functional group (not specifically illustrated in FIG. 8B) corresponding to functional group 16 described with reference to FIGS. 1A-1B and 2A-2F.

While FIGS. 8A-8B illustrate nonlimiting examples including eight functional groups “X” to which respective elongated polymers 13 may be attached, or from which respective elongated polymers 13 may be formed, it will be appreciated that the dendritic core may include any suitable number of functional groups “X,” e.g., two or more, or four or more, illustratively four, six, eight, sixteen, thirty-two, or the like. Branched molecules including desired chemical functional groups are commercially available. For example, FIGS. 9A-9F schematically illustrate example dendritic cores that may be used in the example methods of FIGS. 8A-8B. FIG. 9A illustrates an example 2-arm dendritic core 91. Dendritic core 91 includes first generation focal point 901, illustratively lysine (denoted “K”) which may be used because it has an N-terminal NH₂ and also side chain NH₂ which allows a single K to grow into 2 arms. Focal point 901 is coupled to two functional groups X and to one functional group Y. The squiggle between K and Y in FIG. 9A (and other figures herein) is intended to denote that the coupling between these two elements may not necessarily be direct; however the absence of squiggles between other elements should not be inferred to mean that the coupling between those elements must necessarily be direct.

FIG. 9B illustrates an example 4-arm dendritic core 92, including first generation focal point 901 which is coupled to two second generation focal points 902. Each of second generation focal points 902 of core 92 is coupled to two functional groups X for a total of four functional groups. Additionally, dendritic core 92 is coupled to one functional group Y. FIG. 9C illustrates an example 8-arm dendritic core 93, including first generation focal point 901 which is coupled to two second generation focal points 902. Each of second generation focal points 902 of core 93 is coupled to two third generation focal points 903. Each of third generation focal points 903 is coupled to two functional groups X for a total of eight functional groups. Additionally, dendritic core 93 is coupled to one functional group Y. FIG. 9D illustrates an example 16-arm dendritic core 94, including first generation focal point 901 which is coupled to two second generation focal points 902. Each of second generation focal points 902 of core 93 is coupled to two third generation focal points 903. Each of third generation focal points 903 is coupled to two fourth generation focal points 904, each of which is coupled to additional functional groups X for a total of sixteen functional groups. Additionally, dendritic core 94 is coupled to one functional group Y.

While FIGS. 9A-9D illustrate examples in which outer branches of the dendritic core respectively are coupled to one functional group X and in which inner branches of the dendritic core are coupled to other focal points and not to functional groups, other examples readily may be envisioned. For example, FIG. 9E illustrates an example two-arm dendritic core 95. Dendritic core 95 includes focal point 911 which is coupled to one functional group Y, and is coupled to two additional generation focal points 912. Accordingly, dendritic core 95 is branched at focal point 911. As shown in FIG. 9E, each of the additional focal points 912 is coupled to another such additional focal point, and also is coupled to a functional group X. A plurality of such additional focal points 912 are linearly coupled to one another along each of the two arms 95′, 95′ of dendritic core 95. The end of each arm may include a further focal point 912 which is coupled to two functional groups X. Accordingly, the dendritic core 95 includes a plurality of arms (e.g., two arms), each of which arms includes plurality of functional groups X along that arm.

As another example, FIG. 9F illustrates an example four-arm dendritic core 96. Dendritic core 96 includes focal point 921 which is coupled to one functional group Y, and is coupled to two additional generation focal points 922. As shown in FIG. 9F, each of the additional focal points 922 is coupled to two further focal points 923. Each further focal point 923 is coupled to another such additional focal point, and also is coupled to a functional group X. Accordingly, dendritic core 96 is branched at focal point 921 and at focal points 922. A plurality of such further focal points 923 are linearly coupled to one another along each of the two arms 96′, 96′ of dendritic core 96. The end of each arm may include an additional focal point 924 which is coupled to two functional groups X. Accordingly, the dendritic core 96 includes a plurality of arms (e.g., four arms), each of which arms includes plurality of functional groups X along that arm.

The dendritic core, and the dendrons, may have any suitable respective compositions. In various examples, the dendritic core may include a branched polypeptide or a branched polyester, ester, amide, ethylene glycol, or oligonucleotide. Additionally, or alternatively, in some examples the inert, elongated polymer may include a polypeptide, polyester, polyamide, poly(ethylene glycol), or oligonucleotide.

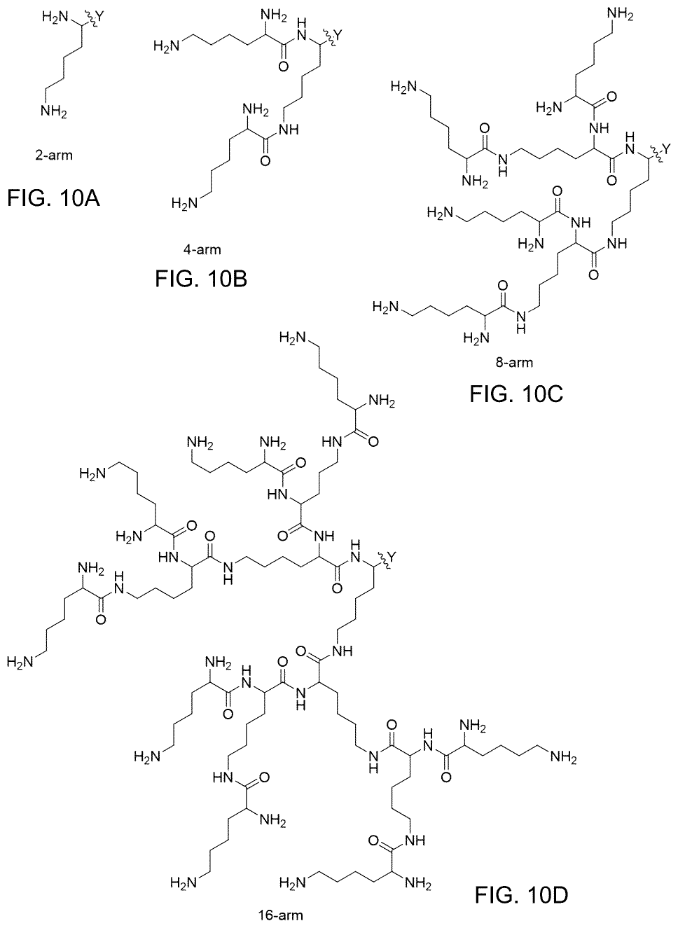

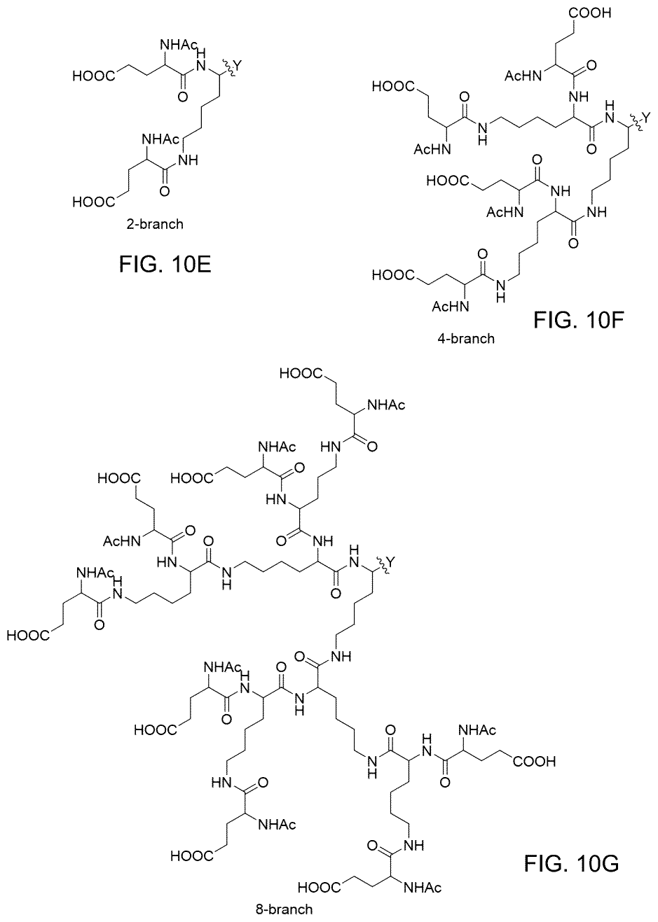

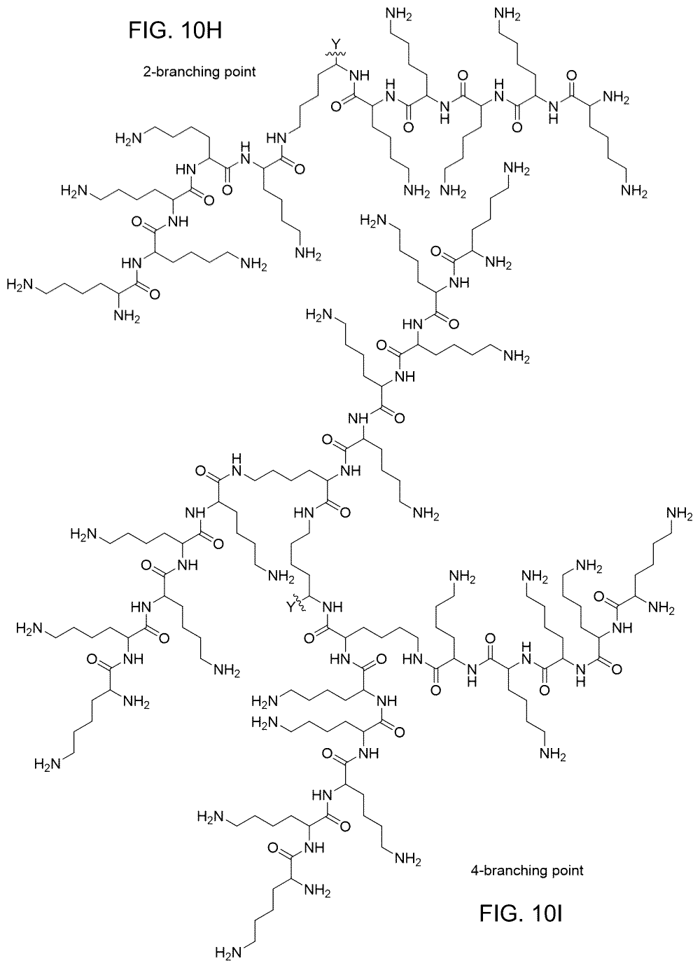

FIGS. 10A-10I schematically illustrate additional example dendritic cores that may be used in the example method of FIGS. 8A-8B. For example, FIG. 10A illustrates a nonlimiting example of a two-arm dendritic core having a configuration similar to that of FIG. 9A, and which is a branched polypeptide that includes two amine groups (—NH₂) corresponding to “X” in FIGS. 8A, and one “Y” group (such as —COOH). FIG. 10B illustrates a nonlimiting example of a four-arm dendritic core having a configuration similar to that of FIG. 9B, and which is a branched polypeptide that includes four “X” amine groups (—NH₂) and one “Y” group (such as —COOH). FIG. 10C illustrates a nonlimiting example of an eight-arm dendritic core having a configuration similar to that of FIG. 9C, and which is a branched polypeptide that includes eight “X” amine groups (—NH₂) and one “Y” group (such as —COOH). FIG. 10D illustrates a nonlimiting example of a sixteen-arm dendritic core having a configuration similar to that of FIG. 9D, and which is branched polypeptide that includes sixteen “X” amine groups (—NH₂) and one “Y” group (such as —COOH). Examples such as illustrated in FIGS. 10A-10D may be particularly suitable for use in examples such as described with reference to FIG. 8A, e.g., may be readily coupled to preformed polymer dendrons using the —NH₂X groups, and readily may be formed using repetitive NH₂ to COOH coupling on a resin/solid support using peptide chemistry, followed by cleavage from the resin and purification to obtain the desired peptide. Traditional peptide synthesis occurs from the C—>N terminal.

As another example, FIG. 10E illustrates a nonlimiting example of a two-arm dendritic core having a configuration similar to that of FIG. 9A, and which is a branched polypeptide that includes two —COOH groups corresponding to “X” in FIGS. 8A-8B and 9A-9D, and one “Y” group (such as —NH₂). FIG. 10F illustrates a nonlimiting example of a four-arm dendritic core having a configuration similar to that of FIG. 9B, and which is a branched polypeptide that includes four “X”-COOH groups and one “Y” group (such as —NH₂). FIG. 10C illustrates a nonlimiting example of an eight-arm dendritic core having a configuration similar to that of FIG. 9C, and which is a branched polypeptide that includes eight “X”-COOH groups and one “Y” group (such as —NH₂). Examples such as illustrated in FIGS. 10E-10G may be particularly suitable for use in examples such as described with reference to FIG. 8A, e.g., may be readily coupled to preformed polymer dendrons using the —COOH X groups. Examples such as illustrated in FIGS. 10E-10G also may be particularly suitable for use in examples such as described with reference to FIG. 8B. For example, a RAFT agent for use in generating dendrons using RAFT polymerization may be coupled to the —COOH X groups. Note that any amines within the branched peptide may be protected (e.g., using acyl groups, as illustrated in FIGS. 10E-10G).

As yet another example, FIG. 10H illustrates a nonlimiting example of a two-arm, two-branching point dendritic core having a configuration similar to that of FIG. 9E, and which is a branched polypeptide that includes twelve amine groups (—NH₂) corresponding to “X” in FIGS. 8A, and one “Y” group (such as —COOH). FIG. 10B illustrates a nonlimiting example of a four-arm, four-branching point dendritic core having a configuration similar to that of FIG. 9F, and which is a branched polypeptide that includes twenty-four “X” amine groups (—NH₂) and one “Y” group (such as —COOH). Examples such as illustrated in FIGS. 10A-10D may be particularly suitable for use in examples such as described with reference to FIG. 8A, e.g., may be readily coupled to preformed polymer dendrons using the —NH₂X groups. The squiggles in FIGS. 10A-10I are intended to represent that the Y group may be coupled directly or indirectly to the rest of the dendritic molecule. For example, Y may be an amino acid residue, or Y may not be an amino acid residue and may be coupled to the rest of the dendritic molecule via an amino acid residue which is represented by the squiggle.

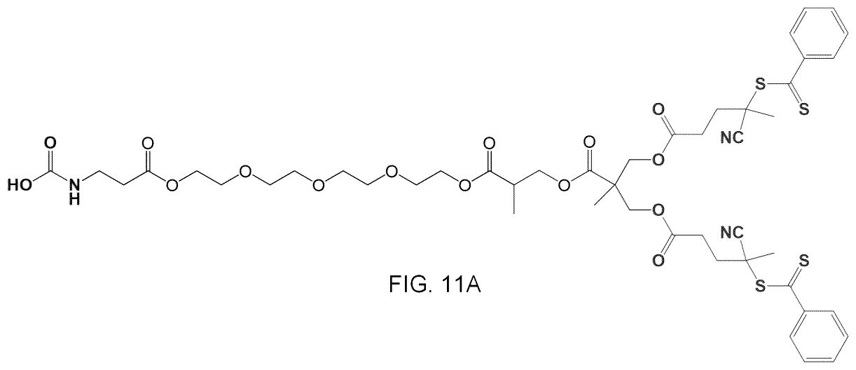

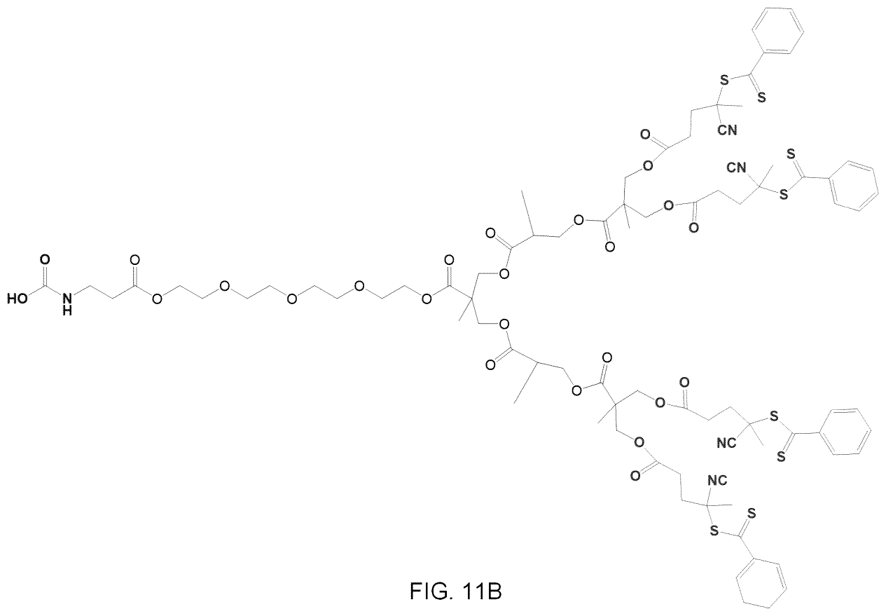

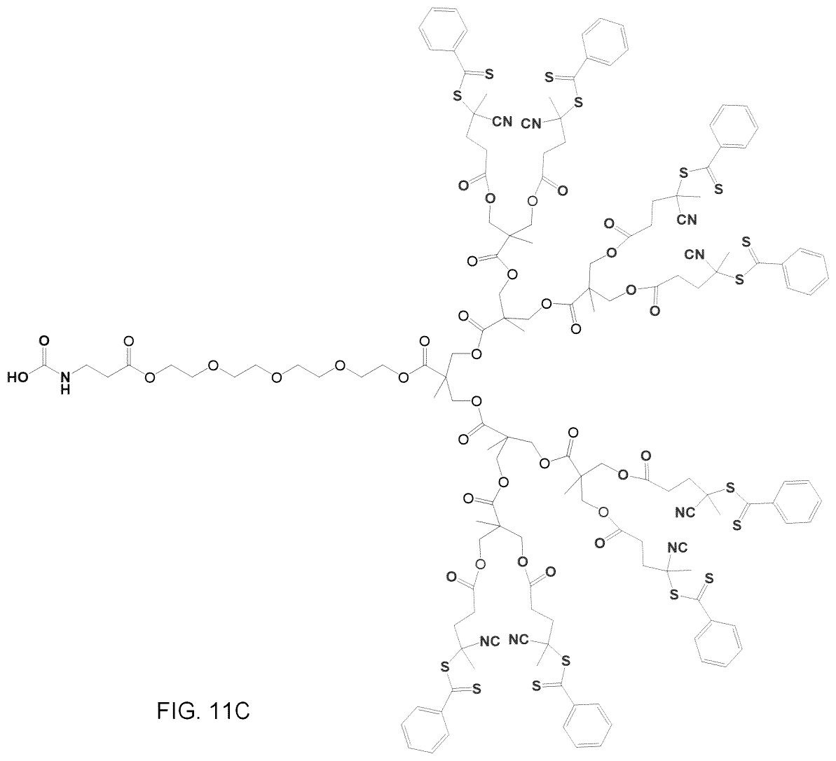

FIGS. 11A-11C schematically illustrate additional example dendritic cores that may be used in the example method of FIGS. 8A-8B. For example, FIG. 11A illustrates a nonlimiting example of a two-arm dendritic core having a configuration similar to that of FIG. 9A, and which is a branched polyester bis-MPA dendron, generation 1 including a carboxyl core, and 2 RAFT chain transfer agent (CTA) end groups. Here, CTA=4-Cyano-4-(phenylcarbonothioylthio)pentanoic acid, although another option is (methylol)propionic acid (MPA). The CTA end groups correspond to “X” in FIG. 8B. The carboxyl core corresponds to the “Y” group. Although X is CTA in the illustrated example, X may be any suitable initiator for controlled polymerization, some examples are RAFT, ATRP, NMP and CCTP. FIG. 11B illustrates a nonlimiting example of a four-arm dendritic core having a configuration similar to that of FIG. 9B, and which is a polyester bis-MPA dendron, generation 2 including a carboxyl core, four CTA end groups with CTA=4-Cyano-4-(phenylcarbonothioylthio)pentanoic acid. FIG. 11C illustrates a nonlimiting example of an eight-arm dendritic core having a configuration similar to that of FIG. 9C, and which is a polyester bis-MPA dendron, generation 3 with carboxyl core and eight CTA end groups with CTA=4-Cyano-4-(phenylcarbonothioylthio)pentanoic acid. Dendritic cores such as described with reference to FIGS. 11A-11C may be commercially available, e.g., from Polymer Factory Sweden AB (Stockholm, Sweden). Examples such as illustrated in FIGS. 11A-11C may be particularly suitable for use in examples such as described with reference to FIG. 8B.

Molecules of elongated polymer 13 illustrated in FIG. 8A may be formed in any suitable manner to include a terminal —COOH group, e.g., using a reversible addition fragmentation chain transfer (RAFT) polymerization technique. The —COOH group of respective polymer molecules 13 may be coupled to the amine groups (or other suitable groups) of the dendritic core in any suitable manner, for example using DMTMM amide-coupling agent, to form dendrons. Optionally, the polymer molecules may include a different, functional end group (such as an alkyne, amine, carboxylic acid, acetylene, N-hydroxy succinimide, biotin, or thiol) corresponding to functional group 16 described with reference to FIG. 1A. For further details regarding RAFT polymerization, including generating functional groups at the end of a polymer formed using RAFT polymerization, see Perrier, “50th anniversary perspective: RAFT polymerization—a user guide,” Macromolecules 50(19): 7433-7447 (2017), the entire contents of which are incorporated by reference herein.

From the foregoing disclosure, it will be appreciated that primers and adapters having any suitable sequences may be used. In one nonlimiting example, amplification primers 131 are P5 amplification primers, and the amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc. Seeding primers 12 may have any suitable sequence which is orthogonal to the sequences of amplification primers 131, 141. In some examples, seeding primers 12 may be PX primers having the sequence AGGAGGAGGAGGAGGAGGAGGAGG (SEQ ID NO: 5), or PY primers having the sequence 5′-GAA GAA GAA GAA GAA GAA GAA GAA GAA GAA-3′ (SEQ ID NO: 6). Seeding adapters 156 may be cPX (also referred to as PX′) primers having the sequence CCTCCTCCTCCTCCTCCTCCTCCT or cPY (PY′) (SEQ ID NO: 7) primers having the sequence 5′-TTC TTC TTC TTC TTC TTC TTC TTC TTC TTC-3′ (SEQ ID NO: 8).

It will further be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Increasing super-Poisson distributions of useful clusters is useful because more functionally monoclonal substrate regions may result in higher quality signal, and thus improved SBS; however, the seeding of target polynucleotides into substrate regions may follow a spatial Poisson distribution, where the trade-off for increasing the number of occupied substrate regions is increasing the number of polyclonal substrate regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present capture primers and orthogonal capture primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp). For further details of amplification processes such as may be used during, and/or are compatible with, operations such as described with reference to FIGS. 2C-2D, see International Patent Application No. PCT/US2022/053005 to Ma et al., now International Patent Publication No. WO2023/114397, filed Dec. 15, 2022 and entitled “Hybrid Clustering,” and International Patent Application No. PCT/EP2023/058307 to Ma et al., now International Patent Publication No. WO2023/187061, filed Mar. 30, 2023 and entitled “Paired-End Resynthesis Using Blocked P5 Primers,” the entire contents of each of which are incorporated by reference herein.

### WORKING EXAMPLES

The following examples are intended to be purely illustrative, and not limiting of the present invention.

These examples highlight the successful synthesis of peptide-based dendritic molecules covalently bonded to a seeding primer (FIGS. 5A and 8), the capture of such dendritic molecules within nanowells following a “head-first” approach (FIGS. 6A-6B), synthesis data and an enhancement of monoclonality compared to control (for the best tile).

### Example 1. Synthesis

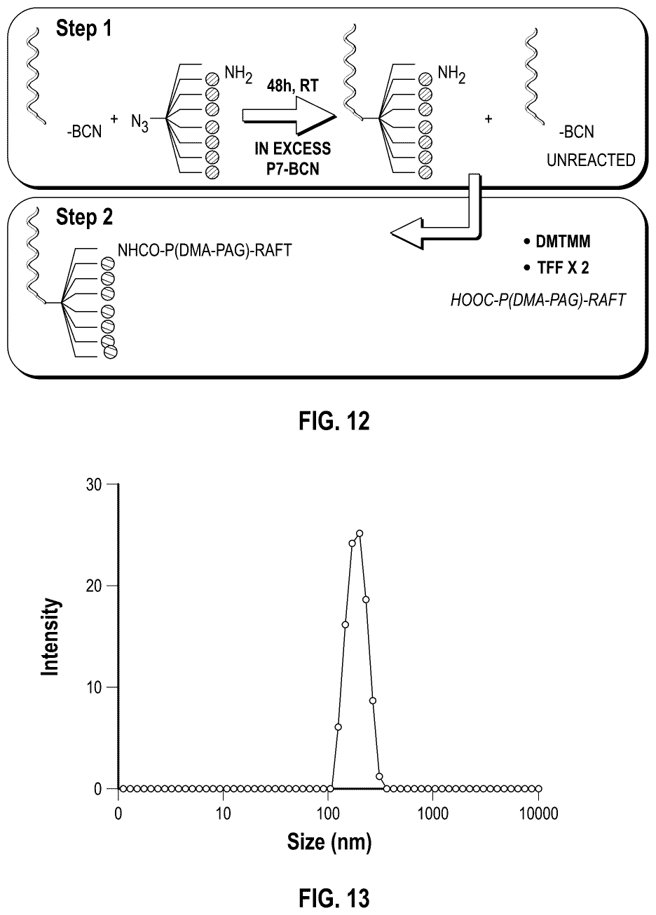

FIG. 12 schematically illustrates a method that was used to prepare dendritic molecules, and FIG. 13 illustrates a dynamic light scattering (DLS) trace from the dendritic molecules prepared in accordance with FIG. 12.

More specifically, FIG. 12 demonstrates a two-step, one-pot synthesis of an oligo-peptide dendritic molecule (OPD). In step 1, a BCN-ended P5 primer was reacted with an N3-terminated 8-arm NH₂ dendritic core at room temperature, in accordance with the reaction between W and Y in FIG. 8. In step 2 of FIG. 12, pre-formed random copolymer-polymer molecules (dimethyl acrylamide-co-propargyl acrylate) (DMA-co-PAG or DMA-PAG) with carboxylic acid end groups (HOOC—P(DMA-co-PAG)-RAFT) are coupled to the amine (—NH₂) end groups of the dendritic core via DMTMM coupling, in accordance with the reaction between X and Z in FIG. 8. After purification via tangential flow filtration (TFF) a 100 kDa molecular weight cutoff (MWCO) membrane, OPDs with an average size of 200 nm were collected, see DLS trace in FIG. 13.

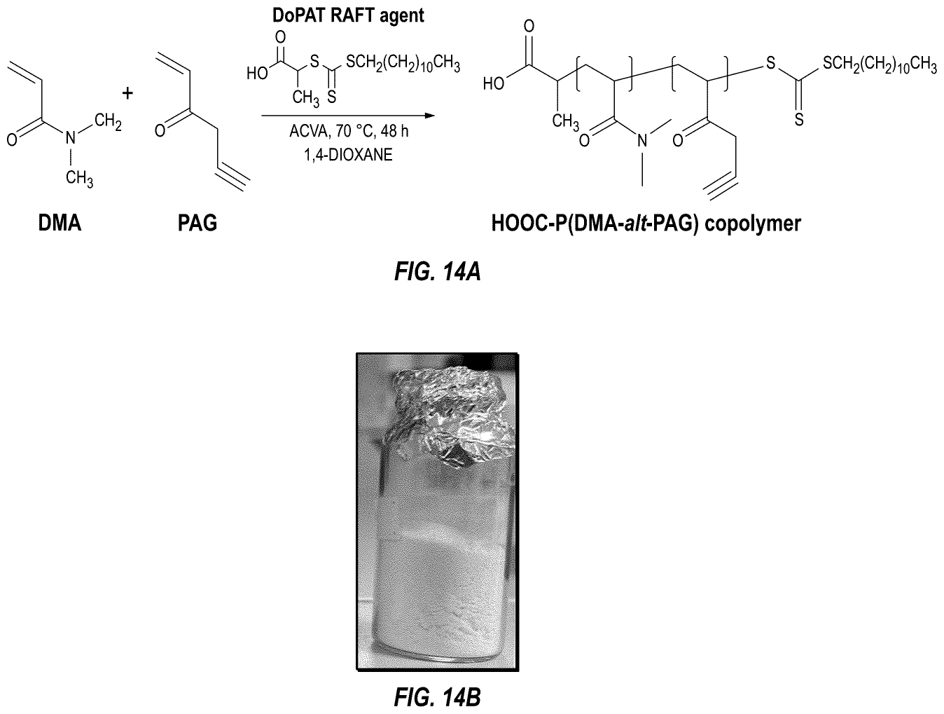

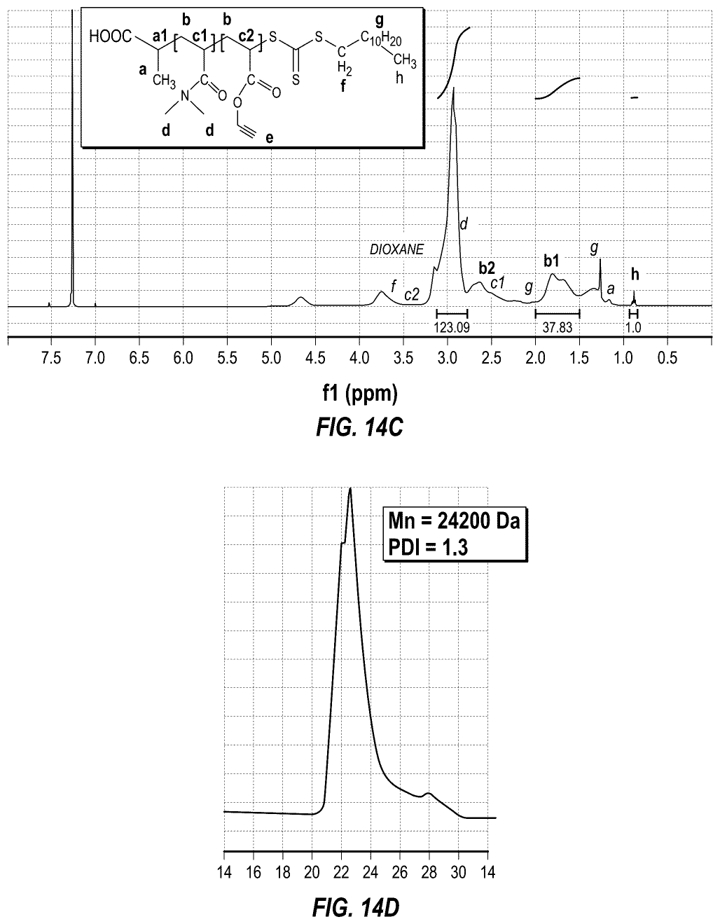

FIG. 14A illustrates reactions in a method with which a functionalized polymer was prepared. FIG. 14B is a photograph of the functionalized polymer prepared in accordance with FIG. 14A. FIG. 14C is a nuclear magnetic resonance (NMR) trace of the functionalized polymer prepared in accordance with FIG. 14A. FIG. 14D is a gel permeation chromatography (GPC) trace of the functionalized polymer prepared in accordance with FIG. 14A, in water. More specifically, the synthesis and characterization of pre-formed polymers HOOC—P(DMA-co-PAG)-RAFT which were used in Step 2 of FIG. 12 is illustrated in FIG. 14A using RAFT polymerization with DoPAT RAFT agent, which carries carboxylic acid functionality. FIG. 14B shows a photograph of the polymer powder. NMR analysis shown in FIG. 14C indicates that the degree of polymerization (DP) was approximately equal to 60. According to GPC analysis, the polymer shows a number average molecular weight of approximately 24 KDa with a PDI of 1.3 (FIG. 14D).

### Example 2. Seeding, Clustering and SBS

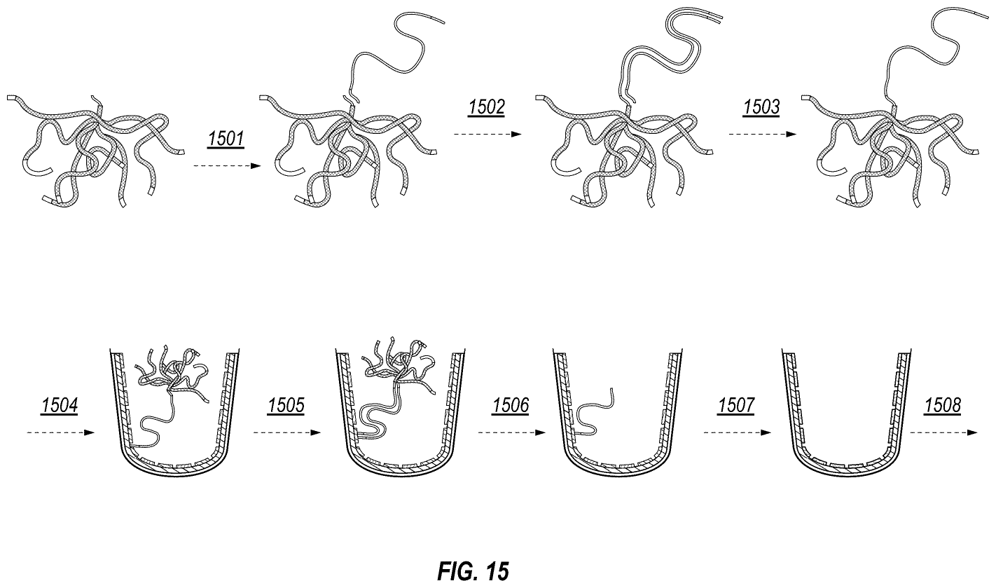

After synthesis, as described in example 1, the OPD was then seeded via the head-first approach (as described with reference to FIGS. 5A-5D and 6A-6F). FIG. 15 schematically illustrates the way a dendritic particle prepared in accordance with FIG. 12 was used to capture and amplify a polynucleotide.

First, PhiX library polynucleotides were seeded onto respective OPDs in solution with the ratio of 1:1 or 2:1 PhiX:OPD (operation 1501). More specifically, the P5 primer attached to the dendritic core of the OPD was hybridized to the cP5 at the end of the PhiX library. DNA chain extension is then carried out for 20 min at 60° C., (operation 1502) followed by de-hybridization in NaOH (operation 1503) and TFF purification using 100 nm pore-size membrane to remove un-hybridized libraries. The OPD carrying PhiX (So called OPD-PhiX) was then seeded into the nanowell of a HiSeq X flowcell via P7-cP7 hybridization (operation 1504), followed by chain extension from grafted PAZAM (operation 1505). The steric hindrance generated by the presence of the OPD in the well inhibited or prevented multiple seeding events to take place within the same nanowell, hence this process was called Size Exclusion Monoclonal Seeding (SEMS). After de-hybridization (operation 1506), the covalently bonded PhiX was then amplified on board (operation 1507) and subjected to sequencing by synthesis (SBS) (operation 1508).

The SBS data show the comparison between seeding of the PhiX directly to the nanowell (control) and capturing of the OPD to the nanowell (NW). SBS was run on a custom 2-channel R/G HiSeq X (HSK191) which allows for the collection of SNR data. This data was recorded from 300 cycles of single read SBS. The successful seeding via SEMS methods, and the validity of the approach were demonstrated through the collection of sequencing data.

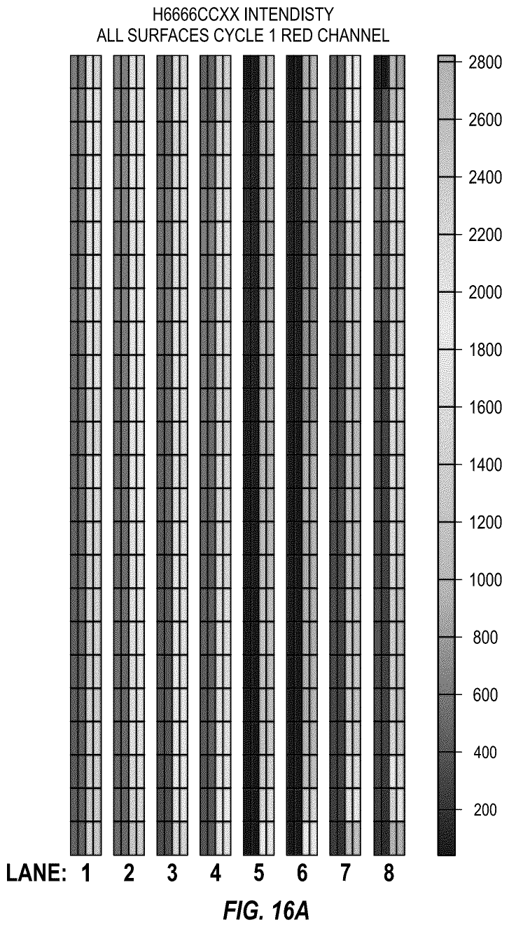

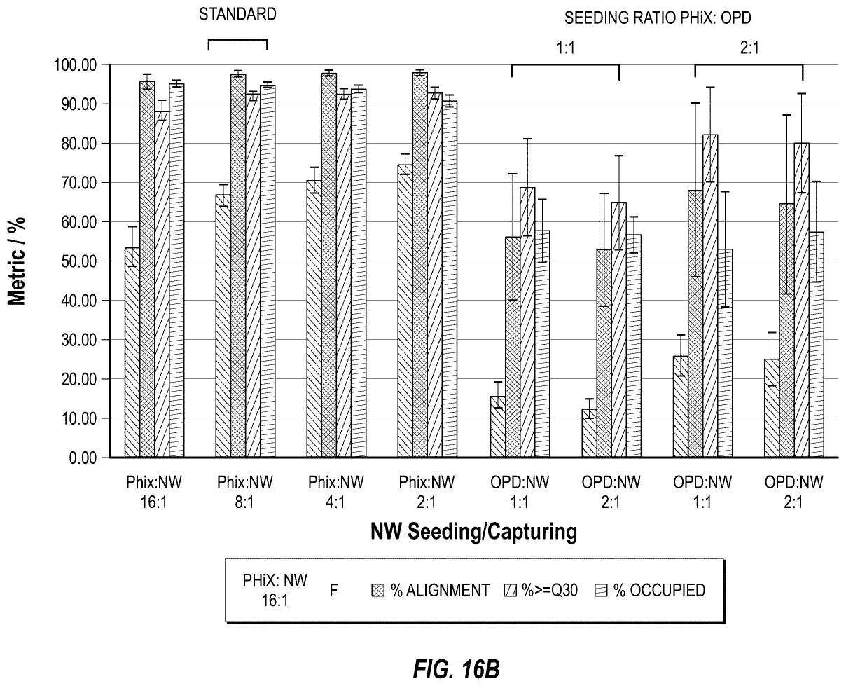

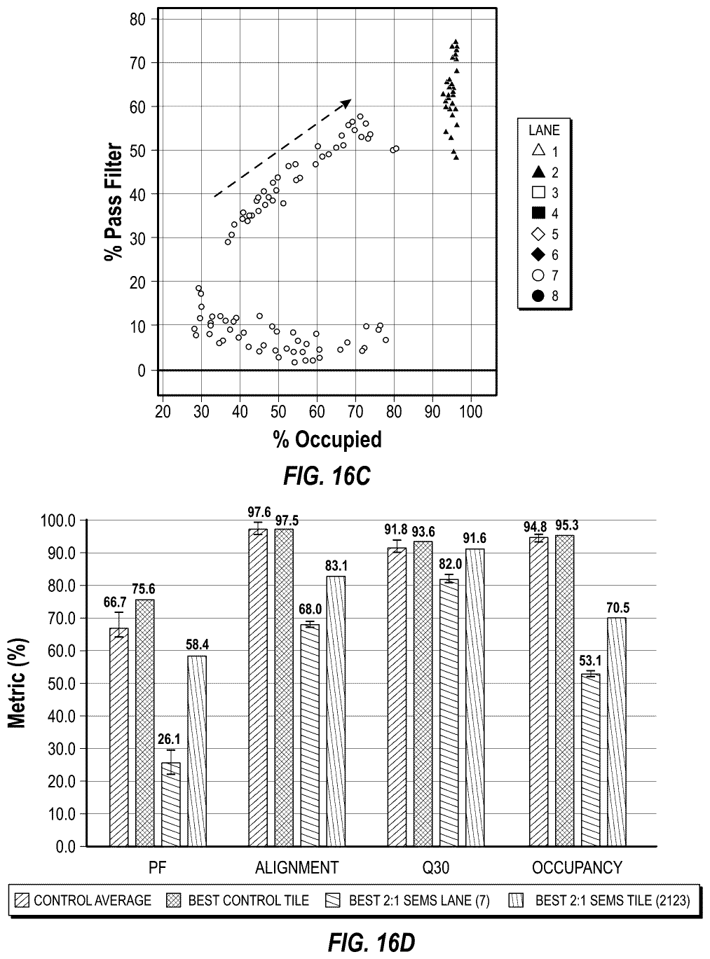

FIGS. 16A-16D are plots illustrating sequencing data obtained using the amplified polynucleotide of FIG. 15. More specifically, FIG. 16A illustrates heatmap C1 intensity for all lanes. Lanes 1-4: Different ratio of PhiX:NW=16:1, 8:1, 4:1 and 2:1, respectively. Lanes 5-8, different ratio of OPDx:NW, X=ratio seeding of PhiX:OPD solution. Lanes 5 & 6, OPD 1:1:NW=1:1 and 2:1 respectively. Lanes 7 & 8, OPD 2:1:NW=1:1 and 2:1 respectively. PhiX:NW=8:1 is of standard seeding. FIG. 16B illustrates lane average SBS metric comparison of different conditions. FIG. 16C illustrates % occupied vs. % Pass Filter plot between PhiX:NW=8:1 (Lane 2) and OPD2:1:NW=1:1 (Lane 7). FIG. 16D illustrates direct comparison of SBS metric of PhiX:NW=8:1 (Lane 2) and OPD2:1:NW=1:1 (Lane 7) with best tile analysis. SBS was run on a custom 2-channel HiSeq X (HSK191) which allows for the collection of SNR data. This data was recorded from 300 cycles of single read SBS. It can be clearly seen in FIG. 16B that with both seeding ratio of PhiX:OPD=1:1 and 2:1, both OPD:NW=1:1 and 2:1 show successful SBS metric. When comparing PhiX:NW=8:1 (Lane 2) and OPD2:1:NW=2:1 (Lane 7), the waterfall plot of % Occupied vs. % PF of OPD2:1:NW=2:1 follows the same trend as that of the standard (FIG. 16C). Closer comparison between these two lanes including the best tiles is shown in FIG. 16D. Although all metrics from the best tile using OPD-SEMS are lower than those from best tile of the standard, all values are of approximately 70-80% of the standard which indicates that the OPDs may be used successfully.

In this section, the data have shown that better clonality and SNR were obtained when comparing the above two lanes: 2 (control lane) and 7 (SEMS seeded lanes).

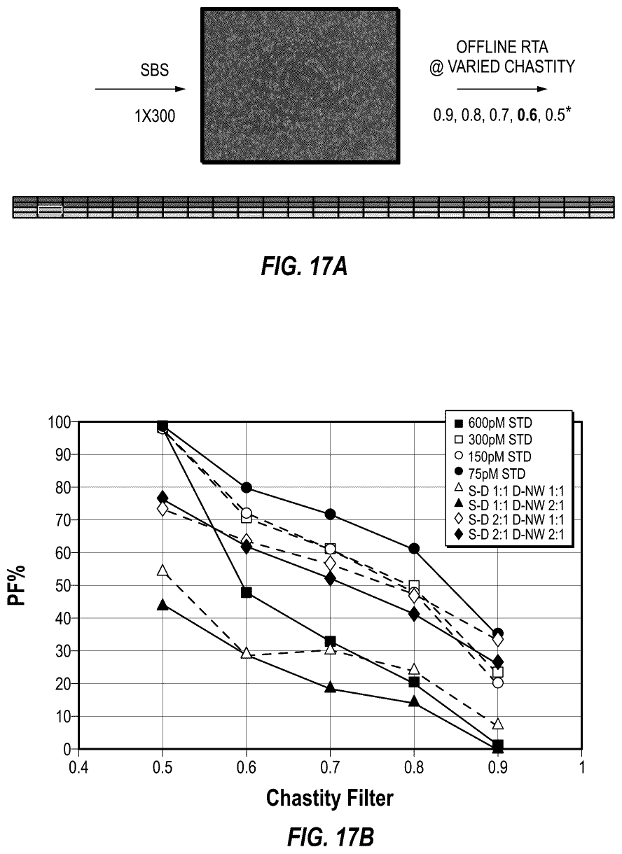

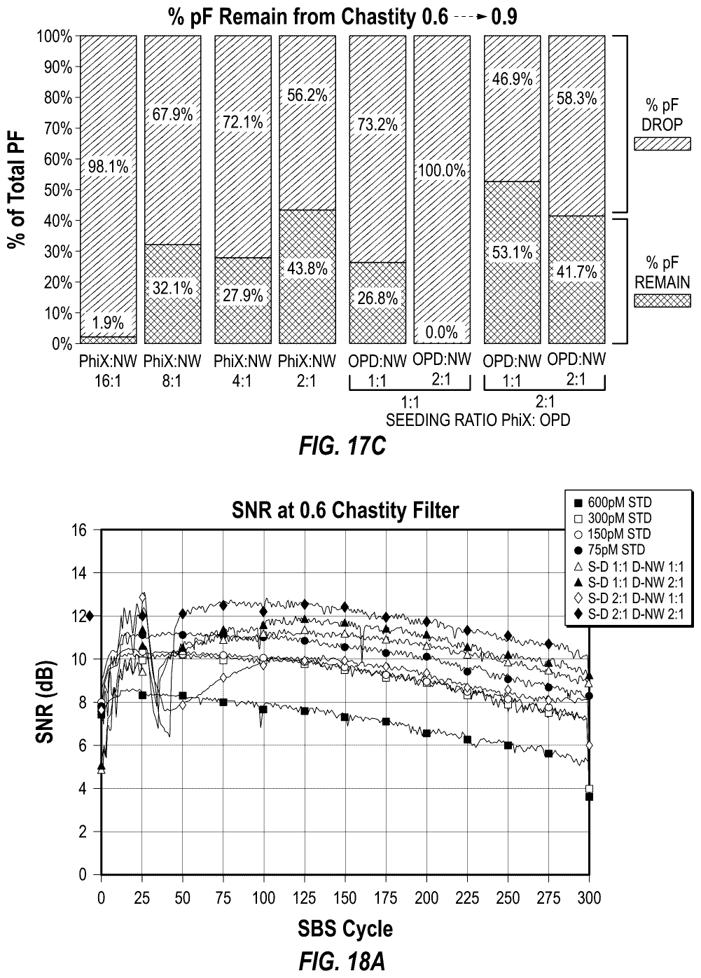

To further assess clonality and signal to noise ratio (SNR), the flowcell (FC) was subjected to a longer run (1×300 cycles) saving the best tile for assessing % PF relationship with Chastity filter (FIGS. 17A-17C). The images saved were then used to run real time analysis (RTA) offline at different Chastity filters recording primary metrics and SNR data each time. % PF (or passing filter), is the percentage of clusters that are taken into account during sequencing. The clusters that do not pass filter usually are too dim or polyclonal or simply empty wells. Chastity is a measure of the clonality of the cluster, and scales from 0.5 to 1. A chastity of 0.5 means that the cluster is split equally between 2 clones. A chastity of 1 means that the cluster is mono-clonal. On most platform the chastity filter is set at 0.6, meaning that each cluster having a score higher than 0.6 will pass the filter. With a score below 0.6 the cluster will be binned and no bases will be called from that cluster during sequencing. The % PF calculations involve the application of a chastity filter to each cluster. Chastity is defined as the ratio of the brightest base intensity divided by the sum of the brightest and second brightest base intensities. Clusters “pass filter” if no more than 1 base call has a chastity value below 0.6 in the first 25 cycles. This filtration process removes the least reliable clusters from the image analysis results.

The relationship between % PF Remain and Chastity can offer an indirect way of assessing clonality. It is expected that at higher Chastity filter, only more monoclonal clusters are selected. Hence, when comparing two clusters, the larger % PF Remain at higher Chastity is a strong indication for one cluster to be more monoclonal than the other. FIGS. 17A-17C are plots illustrating additional sequencing data obtained using the amplified polynucleotide of FIG. 15. More specifically, FIGS. 17A-17C illustrate clonality analysis based on assessing % PF Remain with increasing Chastity filter. FIG. 17A illustrates a workflow of selecting a best tile and re-analysis by offline RTA at different Chastity 0.5, 0.6, 0.7, 0.8 and 0.9 respectively (note that 0.6 is the standard Chastity). FIG. 17B illustrates change of % PF remaining with higher Chastity filter. Squares represent standard seeding with different PhiX:NW, while circles represent SEMS-OPD with different OPDx:NW. X=ratio seeding of PhiX:OPD in solution. Overall, all % PF decreases with increasing Chastity filter. FIG. 17C illustrates % PF remaining when changing from Chastity 0.6 (100%) to Y % at Chastity 0.9. (Orange Region): % PF Remain; (Grey): % PF Drop. In FIG. 17C, Chastity 0.6 is the standard SBS Chastity and all initial % PF at Chastity 0.6 is considered to be 100%. % PF remain when changing from Chastity 0.6 (100%) to Y % at Chastity 0.9 is plotted in FIG. 17C. As can be seen in FIG. 17C, when comparing to lane 2 (Standard, 300 pM, PhiX:NW=8:1, % PF Remain=32.1%) Lanes 7 and 8 show higher % PF Remain (53% and 41%, respectively). This is a strong indication that enhanced clonality has been successfully achieved with both OPD2:1:NW=1:1 and 2:1.

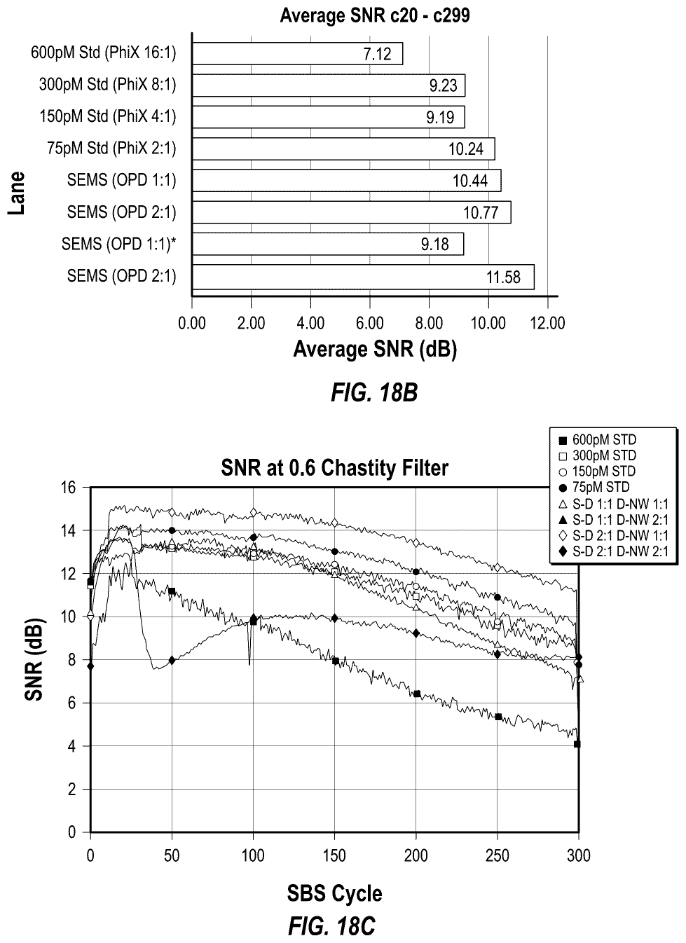

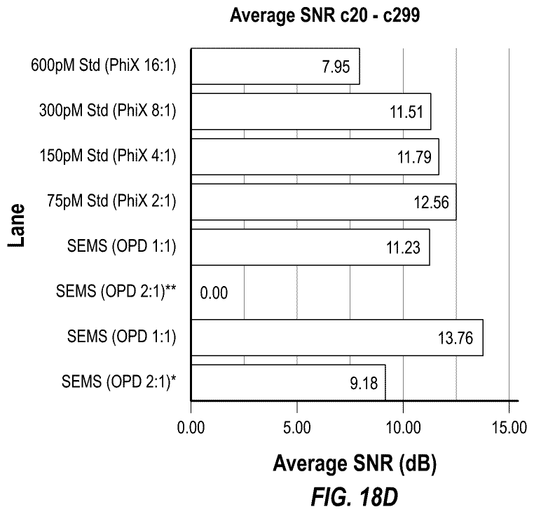

FIGS. 18A-18D are plots illustrating additional sequencing data obtained using the amplified polynucleotide of FIG. 15. More specifically, to further investigate the impact of enhanced monoclonality on SNR, SNR analysis was carried out at both Chastity 0.6 and 0.9. FIGS. 18A-18D show the SNR analysis comparison at Chastity 0.6 (FIGS. 18A-18B) and Chastity 0.9 (FIGS. 18C-18D). In FIG. 18A, the averages SNR of all base-to-base distances are plotted against the number of cycles. FIG. 1B illustrates the average SNR of all cycles between cycles 20-299. Similarly, FIGS. 18C and 18D demonstrate the SNR at Chastity 0.9. In FIG. 18C, the averages SNR of all base-to-base distances are plotted against the number of cycles. FIG. 18D illustrates the average SNR of all cycles between cycles 20-299. In short, SNR (unit dB) can be calculated directly from the distance of a base from another during base calling. SNR can be calculated in pair such as G-T, G-C, C-A & T-A for a certain tile at one certain cycle and Chastity filter. FIGS. 18A-18D demonstrate the SNR value of the same tile of the control lanes (Lane 1,2,3 and 4) and for lane 5, 6, 7 and 8 (said tile was the best tile from lane 7). It was shown that, in the absence of parasite fluorescent signal (noise) from co-cluster (polyclonal) within a well, the SNR is improved. As such, enhanced clonality may result in enhanced SNR as well.

For example, as can be seen in FIG. 18B, the SNR of the control standard increases with the decrease of number of seeded PhiX:NW. Interestingly, SNR values of Lanes 5, 6, 7 and 8 are slightly higher than those of control standards at Chastity 0.6. For the controls, SNR values increase (around 10%) when changing Chastity from 0.6 to 0.9. Furthermore, at Chastity 0.9, decreasing seeding ratio PhiX:NW led to increase in SNR. Also, according to FIG. 17C, for Lane 1-4, decreasing seeding ratio PhiX:NW led to increase of % PF remain (from Chastity 0.6 to 0.9) which is related to monoclonality. These observations strongly suggest that monoclonality does enhance SNR.

For the SEMS-OPD, both FIGS. 18B and 18D suggest that in general, SEMS-OPD approach does offer higher SNR than control standard at both Chastity 0.6 and 0.9. To relate monoclonality to SNR, all SEMS-OPD in Lane 5, 6, 7 and 8 were compared to Lane 2 (300 pM. PhiX:NW=8:1). For Lane 6, at Chastity 0.6, SNR (10.8 dB) value was slightly higher than that of Lane 2 (9.23 dB). Interestingly, at Chastity 0.9 the SNR dropped to SNR=0 dB for Lane 6 while for Lane 2, SNR=11.51 dB. This is in line with the % PF Remain in FIGS. 18B and 18D.

For Lane 7 vs. Lane 2, at Chastity 0.6, both SNR values are similar (9.18 dB and 9.23 dB, respectively). At Chastity 0.9, for Lane 2, SNR=11.54 dB, for Lane 7, SNR=13.76 dB. This observation strongly corroborates the fact that higher % PF remains for Lane 7 (53%, FIG. 17C) cf. % PF for Lane 2 (32.1%, FIG. 17C). % PF remaining is expected to be related to enhanced monoclonality.

Similarly, for Lane 8 vs. Lane 2, at Chastity 0.6, SNR Lane 8 (11.58 dB)>SNR Lane 2 (9.23). At Chastity 0.9, SNR Lane 8 (13.43 dB)>SNR Lane 2 (11.51).

Overall, these data indicate that using SEMS-OPD technique provided herein, an enhancement of SNR and monoclonality have been achieved.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A dendritic molecule for use in capturing a polynucleotide, the dendritic molecule comprising:
a dendritic core;
a seeding primer coupled to the dendritic core; and
a plurality of dendrons, each of the dendrons comprising an inert, elongated polymer comprising a first end coupled to the dendritic core and a second end coupled to a first functional group, wherein the first functional group is to react with a second functional group to form a covalent bond.

2. The dendritic molecule of claim 1, further comprising a polynucleotide comprising a seeding adapter, wherein the seeding adapter is hybridized to the seeding primer.

3. The dendritic molecule of claim 2, wherein the polynucleotide is double-stranded, and the seeding adapter is single-stranded.

4. The dendritic molecule of claim 1, wherein the dendritic core comprises a branched polypeptide, polyester, ester, amide, ethylene glycol, or oligonucleotide.

5. The dendritic molecule of claim 1, wherein the inert, elongated polymer comprises a polypeptide, polyester, polyamide, poly(ethylene glycol), or oligonucleotide.

6. The dendritic molecule of claim 1, wherein the first functional end group comprises an alkyne, amine, carboxylic acid, acetylene, N-hydroxy succinimide, biotin, or thiol.

7. A device, comprising:
a flowcell comprising a plurality of wells, each well comprising a plurality of first functional groups; and
a plurality of dendritic molecules, each dendritic molecule comprising:
a dendritic core;
a seeding primer coupled to the dendritic core; and
a plurality of dendrons, each of the dendrons comprising an inert, elongated polymer comprising a first end coupled to the dendritic core and a second end coupled to a second functional group, wherein the second functional group is to react with one of the first functional groups to form a covalent bond,
wherein at least some of the wells contain a single one of the dendritic molecules.

8. The device of claim 7, wherein the first functional groups form covalent bonds with respective ones of the second functional groups, wherein the covalent bonds immobilize the dendritic molecule within a respective well.

9. (canceled)

10. The device of claim 7, each well further comprising a hydrogel to which the plurality of first functional groups is coupled.

11. The device of claim 7, further comprising a polynucleotide comprising a seeding adapter hybridized to the seeding primer.

12. The device of claim 11, wherein the polynucleotide is double-stranded, and wherein the seeding adapter is single-stranded.

13. The device of claim 7, each well further comprising a plurality of amplification primers, wherein the amplification primers have different sequences than the seeding primer.

14. (canceled)

15. The device of claim 7, further comprising, within wells in which a respective one of the dendritic molecules is disposed and for which the seeding adapter of one of the polynucleotides is hybridized to the seeding primer of that dendritic molecule, a substantially monoclonal cluster of amplicons.

16. The device of claim 7, wherein each of the dendritic molecules contained within a well has a hydrodynamic diameter which is about 60% to about 100% of a diameter of that well.

17. A method of capturing a polynucleotide in a flowcell, the method comprising:
flowing a plurality of dendritic molecules into a flowcell comprising a plurality of wells, each well comprising a plurality of first functional groups,
wherein each dendritic molecule of the plurality comprises:
a dendritic core;
a seeding primer coupled to the dendritic core; and
a plurality of dendrons, each of the dendrons comprising an elongated polymer comprising a first end coupled to the dendritic core and a second end comprising a second functional group;
within at least some of the wells, respectively disposing a respective one of the dendritic molecules within that well;
for each well in which a respective one of the dendritic molecules is disposed, forming covalent bonds between the first functional groups of that well and the second functional groups of that dendritic molecule;
flowing a plurality of polynucleotides into the flowcell, the polynucleotides respectively comprising seeding adapters; and
for each well in which a respective one of the dendritic molecules is disposed, hybridizing a seeding adapter of one of the polynucleotides to the seeding primer of that dendritic molecule.

18. The method of claim 17, wherein the covalent bonds immobilize the dendritic molecule within that well.

19. The method of claim 17, each well further comprising a hydrogel to which the plurality of first functional groups is coupled.

20. The method of claim 17, each well further comprising a plurality of amplification primers, wherein the amplification primers have different sequences than the seeding primer.

21. (canceled)

22. The method of claim 17, further comprising, for each well in which a respective one of the dendritic molecules is disposed and for which the seeding adapter of one of the polynucleotides is hybridized to the seeding primer of that dendritic molecule, using the amplification primers to generate a substantially monoclonal cluster of amplicons of the polynucleotide hybridized to the seeding primer of that dendritic molecule.

23. The method of claim 17, wherein each of the dendritic molecules may have a hydrodynamic diameter which is about 60% to about 100% of a diameter of the wells.

24-37. (canceled)
